SEQUENCE LISTING (14 sequences)

SEQ ID NO: 1 - protein, 397 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSSSTMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - DNA, 1197 bp, synthetic construct
  source 1..1197: mol_type = other DNA
gaattcatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaatcctcgagcaccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 3 - protein, 166 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 4 - protein, 8 aa, synthetic construct
SSSTMVRS

SEQ ID NO: 5 - protein, 223 aa, synthetic construct
VECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - DNA, 504 bp, synthetic construct
  source 1..504: mol_type = other DNA
gaattcatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaa

SEQ ID NO: 7 - DNA, 24 bp, synthetic construct
  source 1..24: mol_type = other DNA
tcctcgagcaccatggttagatct

SEQ ID NO: 8 - DNA, 669 bp, synthetic construct
  source 1..669: mol_type = other DNA
gtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 9 - DNA, 1200 bp, synthetic construct
  source 1..1200: mol_type = other DNA
gaattcatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaatcctcgagcaccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 10 - protein, 375 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSSSTMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 1125 bp, synthetic construct
  source 1..1125: mol_type = other DNA
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaatcctcgagcaccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 12 - protein, 22 aa, synthetic construct
MGRGLLRGLWPLHIVLWTRIAS

SEQ ID NO: 13 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 14 - DNA, 432 bp, synthetic construct
  source 1..432: mol_type = other DNA
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaa

(19) United States
(12) Patent Application Publication
(10) Pub. No.: US 2026/0042817 A1
(43) Pub. Date: Feb. 12, 2026

(54) PET TRACER FOR DETECTION OF TRANSFORMING GROWTH FACTOR BETA

(71) Applicant: Providence Health & Services - Oregon, Portland, OR (US)

(72) Inventors: Kristina Young, Portland, OR (US); Alina Krollenbrock, Milwaukie, OR (US); Alejandro Alice, Portland, OR (US)

(73) Assignee: Providence Health & Services - Oregon, Portland, OR (US)

(21) Appl. No.: 19/150,074

(22) PCT Filed: Jan. 23, 2024
(86) PCT No.: PCT/US2024/012616

§ 371 Date: Jul. 22, 2025

Related U.S. Application Data
(60) Provisional application No. 63/440,768, filed on Jan. 24, 2023.

(51) Int. Cl. C07K 14/71; A61K 51/08

(52) U.S. Cl. CPC C07K 14/71; A61K 51/088; A61K 2123/00; C07K 2319/30

(57) ABSTRACT

Recombinant proteins that specifically bind TGFβ are disclosed. The recombinant proteins include a transforming growth factor beta (TGFβ) binding domain and an antibody Fc fragment. Also disclosed are compositions that include the disclosed recombinant protein, and methods of using such compositions for detecting TGFβ in a subject.

### CROSS REFERENCE TO RELATED APPLICATIONS

This application claims priority to U.S. Provisional Application No. 63/440,768, filed Jan. 24, 2023, which is incorporated by reference in its entirety.

### FIELD

This application relates to recombinant proteins that specifically bind TGFβ and use for detecting TGFβ levels in vivo.

### INCORPORATION OF ELECTRONIC SEQUENCE LISTING

The Sequence Listing is submitted as an XML file named “Sequence.xml,” created on Jan. 22, 2024, 19,587 bytes, which is incorporated by reference herein.

### BACKGROUND

TGFβ is a cytokine with both pro-inflammatory and anti-inflammatory roles, though its anti-inflammatory capacity dominates in the context of tumors. The extracellular concentration of TGFβ is frequently elevated in the tumor microenvironment and is a dominant pathway of immune suppression within this space. TGFβ limits the efficacy not just of immune therapy but also cytotoxic chemotherapy and radiation therapy. Consequently, the presence of high levels of TGFβ in the tumor microenvironment is associated with poor cancer prognosis.

Preliminary evidence demonstrates that TGFβ blockade can improve the outcome of radiation therapy and chemotherapy, but this treatment strategy may be most effective in tumors with high levels of TGFβ. It would therefore be clinically useful to measure TGFβ concentrations in vivo, before and throughout a course of treatment, using an imaging modality such as Positron Emission Tomography (PET). Previous attempts to measure TGFβ levels using radiolabeled antibodies have been limited by prolonged biological elimination and slow radioactive decay and suffer from high background noise. Thus, a TGFβ-specific PET radiotracer with more rapid clearance and decay is desirable.

There are currently no PET radiotracers designed for selective TGFβ detection in clinical use. While ⁸⁹Zr-fresolimumab has been pursued as a potential theranostic (an agent which would both image and eliminate TGFβ in the tumor), this antibody-based approach was found to have several drawbacks. In vivo murine studies were limited by high levels of background noise, and required optimization by imaging 3 days after injection. In addition, there are radiation safety concerns with using ⁸⁹Zr decay due to a relatively high energy 908.97 keV gamma decay and long half-life (78.4 hours). Intensive shielding and long isolated housing of exposed animals is required, making it expensive and less desirable. Finally, ⁸⁹Zr affinity for bone may also impair detection of bony metastases.

### SUMMARY

Active TGFβ binds to the extracellular domain of the TGFβ type II receptor (TβRII), triggering downstream signaling via canonical SMAD and non-canonical pathways. Decoupling the TβRII extracellular domain from the receptor's transmembrane and intracellular domains maintains its ability to bind to TGFβ but prevents downstream intracellular signaling events. Thus, the isolated extracellular domain of TβRII can serve as a TGFβ ‘trap’ by binding to the cytokine, which would otherwise be available to bind to complete receptors and initiate signaling. Here, a TβRII extracellular domain is fused with a solubilizing Fc domain to develop a TGFβ-specific Positron Emission Tomography (PET) radiotracer.

The disclosed radiotracer differs from ⁸⁹Zr-fresolimumab in several significant ways. First, the Fc:TβRII radiotracer utilizes the native TβRII receptor to bind TGFβ, rather than an antibody binding fragment, and TβRII has a high affinity for its ligand TGFβ. Second, the disclosed radiotracer is a recombinant antibody fragment rather than a full-length antibody (like ⁸⁹Zr-fresolimumab). Antibody fragments have many qualities that make them superior for PET radiotracers. Due to their smaller size, antibody fragments can penetrate further into dense tumor tissue than full-length antibodies, leading to better imaging and more complete quantification of the target molecule. Full length antibodies also take much longer to clear from the bloodstream than smaller molecules because they exceed the glomerular filtration threshold, causing them to have a high background signal. This low target to background ratio must often be mitigated by pre-loading with a blocking dose of unlabeled antibody, and repeated imaging is required to build sufficient signal, requiring more time and absorbed radiation dose for the patient. In addition, full length antibodies have a longer biologic half-life resulting in prolonged radioactivity and enhanced radiation safety concerns. In the examples disclosed herein, the IgG2 subclass was selected for the Fc portion of the molecule to facilitate potential clinical translation in part because IgG2 is the IgG subtype with the lowest overall affinity for all major FcγRs, resulting in the lowest predicted background signal caused by FcγR binding. Finally, 8″Zr decay includes a higher energy 908.97 keV gamma decay, while ⁶⁴Cu decays primarily via electron capture, beta-decay, or positron emission. ⁸⁹Zr also has a longer half-life (78.4 h vs 12.7 h) requiring more intensive shielding and longer isolated housing of exposed animals making it more expensive and less desirable. The disclosed Fc:TβRII radiotracer aims to selectively and quantitatively detect TGFβ in vivo, and is designed for improved tumor penetration and pharmacokinetic properties.

Disclosed are recombinant proteins that include a TGF-beta binding domain and an antibody Fc fragment. Also disclosed are recombinant molecules that include (i) a recombinant protein including a TGF-beta binding domain and an antibody Fc fragment, and (ii) a metal ion chelator moiety covalently attached to the recombinant protein. The TGF-beta binding domain includes a portion of a TGF-β type II receptor (TβRII) that specifically binds TGFβ. In some examples, the portion of TβRII is an extracellular domain of TβRII. In some examples, the antibody Fc fragment is an IgG2 Fc fragment. In some examples, the metal ion chelator moiety is a copper chelator, such as 1,4,7-triazacyclononane-1,4,7-triacetic acid (NOTA), 1,4,7,10-tetraazacyclododecane-1,4,7,10-tetraacetic acid (DOTA), or 1,4,7-triazacyclononane,1-glutaric acid-4,7-acetic acid (NODAGA). The disclosure includes cells, such as mammalian cells (e.g., human cells), expressing the disclosed recombinant proteins. The disclosure further includes nucleic acids and vectors encoding the disclosed recombinant proteins, as well as cells expressing such nucleic acids or vectors. Also disclosed are compositions including a disclosed recombinant molecule and a radionuclide bound to the metal ion chelator.

Methods of producing a disclosed recombinant protein, recombinant molecule, or disclosed composition are also included herein. Further disclosed are methods of detecting TGFβ in a subject, including administering a disclosed recombinant protein, recombinant molecule, or disclosed composition to the subject and detecting the recombinant protein, recombinant molecule, or composition in the subject, for example using positron emission tomography (PET).

The foregoing and other features of the disclosure will become more apparent from the following detailed description, which proceeds with reference to the accompanying figures.

### BRIEF DESCRIPTION OF THE DRAWINGS

FIG. 1: shows a diagram of an exemplary complete Fc:TβRII radiotracer, including the TβRII binding domain, human IgG2 structural domain (IgG2 Fc), NOTA chelator group, and ⁶⁴Cu radionuclide. On the left is a depiction of unbound Fc:TβRII radiotracer; on the right is a depiction of Fc:TβRII bound to a TGFβ dimer.

FIGS. 2A-2B show an exemplary nucleic acid sequence (and corresponding amino acid sequence) of an Fc:TβRII recombinant protein including a signal peptide. Residues marked with a double line indicate the TβRII domain. Residues marked with a dotted line indicate spacers, restriction sites, or a stop codon. Unmarked residues indicate the human IgG2 Fc region. The full-length amino acid sequence shown is SEQ ID NO: 1 and the full-length nucleic acid sequence shown is SEQ ID NO: 9 (which is SEQ ID NO: 2 including a stop codon).

FIG. 3 shows a GelRed® stained DNA agarose gel showing the complete Fc:TβRII DNA construct in five E. coli colonies. Lane 1 shows the DNA ladder. Lanes 3-7 show the presence of Fc:TβRII at the predicted size in all tested colonies. Lanes 8-12 show that the TβRII domain is in the correct orientation in colonies 1, 2, 3, and 5.

FIG. 4 shows a Western Blot of Fc:TβRII protein produced by E. coli.

FIG. 5 shows an experimental scheme for monitoring tumor response to radiation therapy. Four cohorts of mice are implanted with tumor cells. When tumors reach 25 mm², cohorts 1 and 2 are imaged with ⁶⁴Cu-Fc:TβRII by PET-MRI. Cohorts 1 and 3 receive radiation therapy (RT) by Small Animal Radiation Research Platform (SARRP) at 10 Gy×2 daily fractions. Seven days after RT for cohorts 1 and 3, cohorts 1 and 2 are re-imaged by PET-MRI.

FIG. 6 shows a Western Blot showing Fc:TβRII protein isolated from the supernatant or cell lysate fractions at 24, 48 and 72 hours.

FIG. 7 shows a Coomassie stain of de-glycosylated (45 kDa) and glycosylated (65 kDa) Fc:TβRII protein purified from HEK 293T cells. Lane numbers are indicated on the x-axis.

FIG. 8 shows a Coomassie stained gel of several batches of Fc:TβRII protein. The dates of the batches are indicated on the x-axis. “Combination” refers to several combined batches prepared prior to the dated batches.

FIG. 9 shows an anti-TβRII Western blot of several batches of Fc:TβRII protein. The dates of the batches are indicated on the x-axis. “Combination” refers to several combined batches prepared prior to the dated batches.

FIG. 10 shows an anti-TGFβ1 ELISA assay for the Fc:TβRII protein. The results show dose-dependent binding of Fc:TβRII to TGFβ with an IC₅₀ of 0.27 μg/mL.

FIG. 11 shows an anti-TGFβ1 ELISA assay of Fc:TβRII protein, NOTA conjugated Fc:TβRII protein (5:1), NOTA conjugated Fc:TβRII protein (10:1), and BMS trap control. The results show unchanged TGFβ binding after NOTA conjugation of Fc:TβRII protein.

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an exemplary amino acid sequence of a recombinant protein that specifically binds TGFβ (Fc:TβRII). This sequence includes a signal peptide, which is removed from the mature protein.

MGRGLLRGLWPLHIVLWTRIASTIPPHVQKSVNNDMIVTDNNGAV
KFPQLCKFCDVRFSTCDNQKSCMSNCSITSICEKPQEVCVAVWRK
NDENITLETVCHDPKLPYHDFILEDAASPKCIMKEKKKPGETFFM
CSCSSDECNDNIIFSEEYNTSNPDLLLVIFQSSSTMVRSVECPPC
PAPPVAGPSVFLFPPKPKDTLMISRTPEVTCVVVDVSHEDPEVQF
NWYVDGMEVHNAKTKPREEQFNSTFRVVSVLTVVHQDWLNGKEYK
CKVSNKGLPAPIEKTISKTKGQPREPQVYTLPPSREEMTKNQVSL
TCLVKGFYPSDIAVEWESNGQPENNYKTTPPMLDSDGSFFLYSKL
TVDKSRWQQGNVFSCSVMHEALHNHYTQKSLSLSPGK

SEQ ID NO: 2 is an exemplary nucleic acid sequence encoding the recombinant protein of SEQ ID NO: 1.

GAATTCATGGGTCGGGGGCTGCTCAGGGGCCTGTGGCCGCTGCAC
ATCGTCCTGTGGACGCGTATCGCCAGCACGATCCCACCGCACGTT
CAGAAGTCGGTTAATAACGACATGATAGTCACTGACAACAACGGT
GCAGTCAAGTTTCCACAACTGTGTAAATTTTGTGATGTGAGATTT
TCCACCTGTGACAACCAGAAATCCTGCATGAGCAACTGCAGCATC
ACCTCCATCTGTGAGAAGCCACAGGAAGTCTGTGTGGCTGTATGG
AGAAAGAATGACGAGAACATAACACTAGAGACAGTTTGCCATGAC
CCCAAGCTCCCCTACCATGACTTTATTCTGGAAGATGCTGCTTCT
CCAAAGTGCATTATGAAGGAAAAAAAAAAGCCTGGTGAGACTTTC
TTCATGTGTTCCTGTAGCTCTGATGAGTGCAATGACAACATCATC
TTCTCAGAAGAATATAACACCAGCAATCCTGACTTGTTGCTAGTC
ATATTTCAATCCTCGAGCACCATGGTTAGATCTGTGGAGTGCCCA
CCTTGCCCAGCACCACCTGTGGCAGGACCTTCAGTCTTCCTCTTC
CCCCCAAAACCCAAGGACACCCTGATGATCTCCAGAACCCCTGAG
GTCACGTGCGTGGTGGTGGACGTGAGCCACGAAGACCCCGAGGTC
CAGTTCAACTGGTACGTGGACGGCATGGAGGTGCATAATGCCAAG
ACAAAGCCACGGGAGGAGCAGTTCAACAGCACGTTCCGTGTGGTC
AGCGTCCTCACCGTCGTGCACCAGGACTGGCTGAACGGCAAGGAG
TACAAGTGCAAGGTCTCCAACAAAGGCCTCCCAGCCCCCATCGAG
AAAACCATCTCCAAAACCAAAGGGCAGCCCCGAGAACCACAGGTG
TACACCCTGCCCCCATCCCGGGAGGAGATGACCAAGAACCAGGTC
AGCCTGACCTGCCTGGTCAAAGGCTTCTACCCCAGCGACATCGCC
GTGGAGTGGGAGAGCAATGGGCAGCCGGAGAACAACTACAAGACC
ACACCTCCCATGCTGGACTCCGACGGCTCCTTCTTCCTCTACAGC
AAGCTCACCGTGGACAAGAGCAGGTGGCAGCAGGGGAACGTCTTC
TCATGCTCCGTGATGCATGAGGCTCTGCACAACCACTACACACAG
AAGAGCCTCTCCCTGTCTCCGGGTAAA

SEQ ID NO: 3 is an exemplary amino acid sequence of a portion of TGFβ type II receptor (TβRII) that specifically binds TGFβ including a signal peptide.

MGRGLLRGLWPLHIVLWTRIASTIPPHVQKSVNNDMIVTDNNGAV
KFPQLCKFCDVRFSTCDNQKSCMSNCSITSICEKPQEVCVAVWRK
NDENITLETVCHDPKLPYHDFILEDAASPKCIMKEKKKPGETFFM
CSCSSDECNDNIIFSEEYNTSNPDLLLVIFQ

SEQ ID NO: 4 is an exemplary amino acid sequence of a spacer.

SSSTMVRS

SEQ ID NO: 5 is an exemplary amino acid sequence of a human IgG2 Fc region including a hinge region, CH2 domain, and CH3 domain.

VECPPCPAPPVAGPSVFLFPPKPKDTLMISRTPEVTCVVVDVSHE
DPEVQFNWYVDGMEVHNAKTKPREEQFNSTFRVVSVLTVVHQDWL
NGKEYKCKVSNKGLPAPIEKTISKTKGQPREPQVYTLPPSREEMT
KNQVSLTCLVKGFYPSDIA VEWESNGQPENNYKTTPPMLDSDGS
FFLYSKLTVDKSRWQQGNVFSCSVMHEALHNHYTQKSLSLSPGK

SEQ ID NO: 6 is an exemplary nucleic acid sequence encoding the amino acid of SEQ ID NO: 3.

GAATTCATGGGTCGGGGGCTGCTCAGGGGCCTGTGGCCGCTGCAC
ATCGTCCTGTGGACGCGTATCGCCAGCACGATCCCACCGCACGTT
CAGAAGTCGGTTAATAACGACATGATAGTCACTGACAACAACGGT
GCAGTCAAGTTTCCACAACTGTGTAAATTTTGTGATGTGAGATTT
TCCACCTGTGACAACCAGAAATCCTGCATGAGCAACTGCAGCATC
ACCTCCATCTGTGAGAAGCCACAGGAAGTCTGTGTGGCTGTATGG
AGAAAGAATGACGAGAACATAACACTAGAGACAGTTTGCCATGAC
CCCAAGCTCCCCTACCATGACTTTATTCTGGAAGATGCTGCTTCT
CCAAAGTGCATTATGAAGGAAAAAAAAAAGCCTGGTGAGACTTTC
TTCATGTGTTCCTGTAGCTCTGATGAGTGCAATGACAACATCATC
TTCTCAGAAGAATATAACACCAGCAATCCTGACTTGTTGCTAGTC
ATATTTCAA

SEQ ID NO: 7 is an exemplary nucleic acid sequence encoding the amino acid of SEQ ID NO: 4.

TCCTCGAGCACCATGGTTAGATCT

SEQ ID NO: 8 is an exemplary nucleic acid sequence encoding the amino acid of SEQ ID NO: 5.

GTGGAGTGCCCACCTTGCCCAGCACCACCTGTGGCAGGACCTTCA
GTCTTCCTCTTCCCCCCAAAACCCAAGGACACCCTGATGATCTCC
AGAACCCCTGAGGTCACGTGCGTGGTGGTGGACGTGAGCCACGAA
GACCCCGAGGTCCAGTTCAACTGGTACGTGGACGGCATGGAGGTG
CATAATGCCAAGACAAAGCCACGGGAGGAGCAGTTCAACAGCACG
TTCCGTGTGGTCAGCGTCCTCACCGTCGTGCACCAGGACTGGCTG
AACGGCAAGGAGTACAAGTGCAAGGTCTCCAACAAAGGCCTCCCA
GCCCCCATCGAGAAAACCATCTCCAAAACCAAAGGGCAGCCCCGA
GAACCACAGGTGTACACCCTGCCCCCATCCCGGGAGGAGATGACC
AAGAACCAGGTCAGCCTGACCTGCCTGGTCAAAGGCTTCTACCCC
AGCGACATCGCCGTGGAGTGGGAGAGCAATGGGCAGCCGGAGAAC
AACTACAAGACCACACCTCCCATGCTGGACTCCGACGGCTCCTTC
TTCCTCTACAGCAAGCTCACCGTGGACAAGAGCAGGTGGCAGCAG
GGGAACGTCTTCTCATGCTCCGTGATGCATGAGGCTCTGCACAAC
CACTACACACAGAAGAGCCTCTCCCTGTCTCCGGGTAAA

SEQ ID NO: 9 is an exemplary nucleic acid sequence encoding the recombinant protein of SEQ ID NO: 1 including a stop codon.

GAATTCATGGGTCGGGGGCTGCTCAGGGGCCTGTGGCCGCTGCAC
ATCGTCCTGTGGACGCGTATCGCCAGCACGATCCCACCGCACGTT
CAGAAGTCGGTTAATAACGACATGATAGTCACTGACAACAACGGT
GCAGTCAAGTTTCCACAACTGTGTAAATTTTGTGATGTGAGATTT
TCCACCTGTGACAACCAGAAATCCTGCATGAGCAACTGCAGCATC
ACCTCCATCTGTGAGAAGCCACAGGAAGTCTGTGTGGCTGTATGG
AGAAAGAATGACGAGAACATAACACTAGAGACAGTTTGCCATGAC
CCCAAGCTCCCCTACCATGACTTTATTCTGGAAGATGCTGCTTCT
CCAAAGTGCATTATGAAGGAAAAAAAAAAGCCTGGTGAGACTTTC
TTCATGTGTTCCTGTAGCTCTGATGAGTGCAATGACAACATCATC
TTCTCAGAAGAATATAACACCAGCAATCCTGACTTGTTGCTAGTC
ATATTTCAATCCTCGAGCACCATGGTTAGATCTGTGGAGTGCCCA
CCTTGCCCAGCACCACCTGTGGCAGGACCTTCAGTCTTCCTCTTC
CCCCCAAAACCCAAGGACACCCTGATGATCTCCAGAACCCCTGAG
GTCACGTGCGTGGTGGTGGACGTGAGCCACGAAGACCCCGAGGTC
CAGTTCAACTGGTACGTGGACGGCATGGAGGTGCATAATGCCAAG
ACAAAGCCACGGGAGGAGCAGTTCAACAGCACGTTCCGTGTGGTC
AGCGTCCTCACCGTCGTGCACCAGGACTGGCTGAACGGCAAGGAG
TACAAGTGCAAGGTCTCCAACAAAGGCCTCCCAGCCCCCATCGAG
AAAACCATCTCCAAAACCAAAGGGCAGCCCCGAGAACCACAGGTG
TACACCCTGCCCCCATCCCGGGAGGAGATGACCAAGAACCAGGTC
AGCCTGACCTGCCTGGTCAAAGGCTTCTACCCCAGCGACATCGCC
GTGGAGTGGGAGAGCAATGGGCAGCCGGAGAACAACTACAAGACC
ACACCTCCCATGCTGGACTCCGACGGCTCCTTCTTCCTCTACAGC
AAGCTCACCGTGGACAAGAGCAGGTGGCAGCAGGGGAACGTCTTC
TCATGCTCCGTGATGCATGAGGCTCTGCACAACCACTACACACAG
AAGAGCCTCTCCCTGTCTCCGGGTAAATGA

SEQ ID NO: 10 is an exemplary amino acid sequence of a mature recombinant protein that specifically binds TGFβ (Fc:TβRII).

TIPPHVQKSVNNDMIVTDNNGAVKFPQLCKFCDVRFSTCDNQKSC
MSNCSITSICEKPQEVCVAVWRKNDENITLETVCHDPKLPYHDFI
LEDAASPKCIMKEKKKPGETFFMCSCSSDECNDNIIFSEEYNTSN
PDLLLVIFQSSSTMVRSVECPPCPAPPVAGPSVFLFPPKPKDTLM
ISRTPEVTCVVVDVSHEDPEVQFNWYVDGMEVHNAKTKPREEQFN
STFRVVSVLTVVHQDWLNGKEYKCKVSNKGLPAPIEKTISKTKGQ
PREPQVYTLPPSREEMTKNQVSLTCLVKGFYPSDIAVEWESNGQP
ENNYKTTPPMLDSDGSFFLYSKLTVDKSRWQQGNVFSCSVMHEAL
HNHYTQKSLSLSPGK

SEQ ID NO: 11 is an exemplary nucleic acid sequence encoding the recombinant protein of SEQ ID NO: 10.

ACGATCCCACCGCACGTTCAGAAGTCGGTTAATAACGACATGATA
GTCACTGACAACAACGGTGCAGTCAAGTTTCCACAACTGTGTAAA
TTTTGTGATGTGAGATTTTCCACCTGTGACAACCAGAAATCCTGC
ATGAGCAACTGCAGCATCACCTCCATCTGTGAGAAGCCACAGGAA
GTCTGTGTGGCTGTATGGAGAAAGAATGACGAGAACATAACACTA
GAGACAGTTTGCCATGACCCCAAGCTCCCCTACCATGACTTTATT
CTGGAAGATGCTGCTTCTCCAAAGTGCATTATGAAGGAAAAAAAA
AAGCCTGGTGAGACTTTCTTCATGTGTTCCTGTAGCTCTGATGAG
TGCAATGACAACATCATCTTCTCAGAAGAATATAACACCAGCAAT
CCTGACTTGTTGCTAGTCATATTTCAATCCTCGAGCACCATGGTT
AGATCTGTGGAGTGCCCACCTTGCCCAGCACCACCTGTGGCAGGA
CCTTCAGTCTTCCTCTTCCCCCCAAAACCCAAGGACACCCTGATG
ATCTCCAGAACCCCTGAGGTCACGTGCGTGGTGGTGGACGTGAGC
CACGAAGACCCCGAGGTCCAGTTCAACTGGTACGTGGACGGCATG
GAGGTGCATAATGCCAAGACAAAGCCACGGGAGGAGCAGTTCAAC
AGCACGTTCCGTGTGGTCAGCGTCCTCACCGTCGTGCACCAGGAC
TGGCTGAACGGCAAGGAGTACAAGTGCAAGGTCTCCAACAAAGGC
CTCCCAGCCCCCATCGAGAAAACCATCTCCAAAACCAAAGGGCAG
CCCCGAGAACCACAGGTGTACACCCTGCCCCCATCCCGGGAGGAG
ATGACCAAGAACCAGGTCAGCCTGACCTGCCTGGTCAAAGGCTTC
TACCCCAGCGACATCGCCGTGGAGTGGGAGAGCAATGGGCAGCCG
GAGAACAACTACAAGACCACACCTCCCATGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACACAGAAGAGCCTCTCCCTGTCTCCGGGTAAA

SEQ ID NO: 12 is an exemplary signal peptide sequence.

MGRGLLRGLWPLHIVLWTRIAS

SEQ ID NO: 13 is an exemplary amino acid sequence of a portion of TGFβ type II receptor (TβRII) that specifically binds TGFβ.

TIPPHVQKSVNNDMIVTDNNGAVKFPQLCKFCDVRFSTCDNQKSC
MSNCSITSICEKPQEVCVAVWRKNDENITLETVCHDPKLPYHDFI
LEDAASPKCIMKEKKKPGETFFMCSCSSDECNDNIIFSEEYNTSN
PDLLLVIFQ

SEQ ID NO: 14 is an exemplary nucleic acid sequence encoding the amino acid of SEQ ID NO: 13.

ACGATCCCACCGCACGTTCAGAAGTCGGTTAATAACGACATGATA
GTCACTGACAACAACGGTGCAGTCAAGTTTCCACAACTGTGTAAA
TTTTGTGATGTGAGATTTTCCACCTGTGACAACCAGAAATCCTGC
ATGAGCAACTGCAGCATCACCTCCATCTGTGAGAAGCCACAGGAA
GTCTGTGTGGCTGTATGGAGAAAGAATGACGAGAACATAACACTA
GAGACAGTTTGCCATGACCCCAAGCTCCCCTACCATGACTTTATT
CTGGAAGATGCTGCTTCTCCAAAGTGCATTATGAAGGAAAAAAAA
AAGCCTGGTGAGACTTTCTTCATGTGTTCCTGTAGCTCTGATGAG
TGCAATGACAACATCATCTTCTCAGAAGAATATAACACCAGCAAT
CCTGACTTGTTGCTAGTCATATTTCAA

### DETAILED DESCRIPTION

### Terms

Unless otherwise noted, all technical and scientific terms used herein have the same meaning as commonly understood by one of ordinary skill in the art to which this disclosure belongs. Definitions of common terms in molecular biology may be found in Lewin's Genes XII, ed. Krebs et al., Jones and Bartlett Publishers, 2017 (ISBN 1284143023, 9781284104493); and George P. Rédei, Encyclopedic Dictionary of Genetics, Genomics, Proteomics and Informatics, 3^{rd} Edition, Springer, 2008 (ISBN: 1402067534), and other similar references. In case of conflict, the present specification, including explanations of terms, will control.

Although methods and materials similar or equivalent to those described herein can be used in the practice or testing of the present disclosure, suitable methods and materials are described below. The materials, methods, and examples are illustrative and not intended to be limiting.

The singular terms “a,” “an,” and “the” include plural referents unless the context clearly indicates otherwise. “Comprising A or B” means including A, or B, or A and B. Unless otherwise indicated, the term “about” encompasses a range of 10% of a reference value (e.g., “about 100” refers to a range of 90 to 110). It is further understood that all base sizes or amino acid sizes, and all molecular weight or molecular mass values, given for nucleic acids or polypeptides are approximate, and are provided for description. To facilitate review of the various aspects of the disclosure, the following explanations of specific terms are provided:

Administration: To provide or give a subject an agent by any effective route. Administration can be local or systemic. Exemplary routes of administration include, but are not limited to, oral, injection (such as subcutaneous, intramuscular, intradermal, intraperitoneal, intrahepatic, percutaneous (into the liver), and intravenous), sublingual, rectal, transdermal (for example, topical), intranasal, vaginal, and inhalation routes. In some examples, administration is by injection (e.g., intravenous injection).

Antibody: A polypeptide used by the immune system to recognize antigens; also known as an “immunoglobulin.” Mammalian immunoglobulins are composed of a heavy (H) chain and a light (L) chain, each of which has a variable region, termed the variable heavy (V_{H}) region and the variable light (V_{L}) region, respectively. The fragment antigen-binding region (Fab) region contains the V_{H} and V_{L} regions that define the antigen binding site. In contrast, the fragment crystallizable (Fc) region of an antibody (sometimes referred to as the “tail” region of an antibody) is generated entirely from the heavy chain (H) constant region and does not bind antigens. As used herein, an “antibody Fc fragment” refers to a portion or complete Fc region of an antibody. An antibody Fc fragment can include a CH2 domain, CH3 domain, and/or a hinge region. Fc fragments can self-dimerize via hydrophobic contacts in CH2 domains and link covalently via disulfide bonds in the hinge region. In some cases, an Fc fragment is used to solubilize a recombinant protein.

There are five main heavy chain classes (or isotypes) of mammalian immunoglobulins, which determine the functional activity of an antibody molecule: IgM, IgD, IgG, IgA and IgE. In some examples, the disclosed recombinant protein includes an IgG Fc domain. Antibodies also include genetically engineered forms, such as chimeric antibodies (such as humanized murine antibodies) or heteroconjugate antibodies (such as bispecific antibodies).

Cancer: A malignant tumor characterized by abnormal or uncontrolled cell growth. Other features often associated with cancer include metastasis, interference with the normal functioning of neighboring cells, release of cytokines or other secretory products at abnormal levels, suppression or aggravation of inflammatory or immunological response, invasion of surrounding or distant tissues or organs, such as lymph nodes, etc. “Metastatic” refers to cancer cells that have left the original tumor site and migrated to other parts of the body, for example via the bloodstream or lymph system. In some examples, the cancer is a TGFβ-expressing cancer or cancer that causes elevated TGFβ levels.

Conservative Variant: An amino acid sequence containing at least one conservative amino acid substitution. Conservative substitutions are substitutions of an amino acid with a chemically similar amino acid, and thus the substitution is not expected to substantially affect or decrease a function of a protein (such as the ability of a recombinant protein to bind TGFβ). The following six groups are examples of amino acids that are considered conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Control: A reference standard. Controls can be used to determine whether a measured parameter is relatively increased (elevated) or decreased (suppressed). In some examples of the present disclosure, the measured parameter is TGFβ levels in a subject or sample. In some examples, the TGFβ levels are increased or elevated relative to a control (e.g., a baseline measurement or “healthy” reference standard). In some examples, a “healthy” or normal level of TGFβ is about 19.95 pg/mL (for example, in serum). In some examples, an elevated level of TGFβ is greater than 19.95 pg/mL (for example, in serum). In some examples, the control is a negative control, such as a healthy subject (e.g., a subject not having cancer). In other examples, the control is a historical control or standard reference value or range of values (such as a group of samples from subjects with a known diagnosis and/or outcome, or a group of samples that represent baseline or normal values). A practitioner can readily determine a suitable control/reference standard.

Detect: To identify the presence of a target substance. Detection can be in vivo (e.g., a positron emission tomography scan of a subject) or in vitro (e.g., detection of a target substance in a sample). In some examples of the present disclosure, the presence of TGFβ is detected in vivo using a composition disclosed herein and positron emission tomography (PET).

Effective Amount: The amount of an agent that is sufficient to achieve a desired result. For example, in some examples of the present disclosure, an effective amount of a composition disclosed herein is an amount sufficient to visualize TGFβ in a subject through PET imaging.

Increase or Decrease: A positive or negative change, respectively, relative to a reference or control. An increase is a positive change, such as an increase at least 25%, at least 50%, at least 75%, at least 100%, at least 200%, at least 300%, at least 400%, or at least 500% relative to a suitable control. For example, an increase can be about 25 to 500%, about 25 to 400%, about 25 to 300%, about 25 to 200%, about 25 to 100%, about 25 to 75%, about 25 to 50%, about 50 to 500%, about 75 to 500%, about 100 to 500%, about 200 to 500%, about 300 to 500%, about 400 to 500%, about 50 to 100%, about 50 to 200%, about 50 to 300%, about 50 to 400%, about 50 to 500%, about 100 to 200%, about 100 to 300%, about 100 to 400%, about 100 to 500%, or about 250 to 500%. A decrease is a negative change, such as a decrease of at least 20%, at least 25%, at least 50%, at least 75%, at least 80%, at least 90%, at least 95%, at least 98%, at least 99%, or at least 100% decrease relative to a suitable control. For example, a decrease can be about 25 to 100%, about 25 to 98%, about 25 to 95%, about 25 to 90%, about 25 to 80%, about 25 to 75%, about 25 to 50%, about 50 to 100%, about 75 to 100%, about 90 to 100%, about 95 to 100%, about 98 to 100%, about 99 to 100%, about 50 to 75%, about 50 to 80%, about 50 to 90%, about 50 to 95%, about 50 to 98%, about 75 to 80%, about 75 to 90%, about 75 to 95%, or about 75 to 98%.

Isolated or purified: A biological component, such as a nucleic acid or protein, that has been substantially separated or purified away from other biological components in the environment (such as a cell) in which the component occurs, e.g., other chromosomal and extra-chromosomal DNA and RNA, proteins and cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

The term does not require absolute purity; rather, it is intended as a relative term. Thus, for example, an isolated or purified protein, nucleic acid, or cell preparation is one in which the protein, nucleic acid, or cell is more enriched than the protein, nucleic acid, or cell is in its initial environment. In one example, a preparation is purified such that the protein, nucleic acid, or cell represents at least 50% of the total content of the preparation. In another example, a substantially purified protein or nucleic acid is at least 60% pure, for example, at least 70%, 80%, 90%, 95% or 98% pure. Thus, in one specific, non-limiting example, a substantially purified protein or nucleic acid is 90% free of other components.

Label: A compound or composition that is conjugated directly or indirectly to another molecule (such as a nucleic acid molecule) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable).

Metal ion chelator: A chemical compound that reacts with metal ions to form stable, water-soluble metal complexes. Chelation typically involves the formation or presence of two or more separate coordinate bonds between a polydentate ligand and a single central metal atom. Common metal ion targets include zirconium, iron, copper, mercury, and lead. Exemplary copper chelators include: 1,4,7-triazacyclononane-1,4,7-triacetic acid (NOTA), 1,4,7,10-tetraazacyclododecane-1,4,7,10-tetraacetic acid (DOTA), and 1,4,7-triazacyclononane, 1-glutaric acid-4,7-acetic acid (NODAGA).

Operably linked: A first nucleic acid or amino acid sequence is operably linked with a second nucleic acid or amino acid sequence when the first sequence is placed in a functional relationship with the second sequence. In one example, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical Composition: A composition suitable for pharmaceutical (for example, therapeutic or diagnostic) use in a subject. A pharmaceutical composition comprises an effective amount of an agent (e.g., a radiotracer that specifically binds TGFβ) and a pharmaceutically acceptable carrier.

Pharmaceutically Acceptable Carrier: Compositions and formulations suitable for pharmaceutical delivery. Suitable pharmaceutically acceptable carriers have been described, for example, in Remington's Pharmaceutical Sciences, 23rd Edition, Academic Press, Elsevier, (2020). In general, the nature of the carrier will depend on the particular mode of administration being employed. For instance, intravenous injection formulations usually include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Positron Emission Tomography (PET): A noninvasive diagnostic imaging technique in which substances (radiotracers) containing positron-emitting radionuclides are introduced into a subject, often by intravenous injection. The subject is then scanned and the radiotracer is detected by gamma rays produced by the radionuclides.

Radionuclide: A radioactive nuclide. Exemplary radionuclides include copper-64 (⁶⁴Cu), copper-61 (⁶¹Cu), copper-60 (⁶⁰Cu), zirconium-89 (⁸⁹Zr), gallium-66 (⁶⁶Ga), gallium-68, yttrium-86 (⁸⁶Y), scandium-44, manganese-52, and technetium-94m (^{99m}Tc).

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

Sequence identity: The degree of similarity between amino acid (or nucleotide) sequences. Sequence identity is frequently measured in terms of percent identity (or similarity); the higher the percentage, the more similar the two sequences are. Methods of alignment of sequences for comparison have been described. An exemplary tool that can be used to calculate sequence identity is the NCBI Basic Local Alignment Search Tool (BLAST) (Altschul et al., J. Mol. Biol. 215:403, 1990), which is available from several sources, including the National Center for Biotechnology Information (NCBI, Bethesda, MD). A description of how to determine sequence identity using this program is available on the NCBI website. Unless otherwise specified, sequence identity is determined from an alignment to the reference sequence using NCBI Blast set to default parameters.

Specifically binds: A protein that “specifically binds” a target preferentially binds the target and does not bind in a significant amount any other proteins present in the sample or subject. Binding specificity can be determined using standard methods, for example, by immunoassay (e.g., enzyme-linked immunoassay (ELISA)). In some examples, the recombinant protein or recombinant molecule disclosed herein specifically bind TGFβ.

Subject: A living multi-cellular vertebrate organism, a category that includes mammals. In some examples, the subject is a mammal, for example, a human, non-human primate, cat, dog, or rodent. In a non-limiting example, the subject is a human.

TGFβ: Transforming growth factor beta (TGFβ or TGF-beta) is a multipotent cytokine that can either promote or inhibit cell proliferation depending on the spatial and temporal circumstances. Before a tumor becomes invasive, TGFβ is predominantly tumor-suppressive due to growth inhibitory effects on cyclin-dependent kinase inhibitors 1A and 2A. In established tumors, the role of TGFβ switches to support tumor growth. Canonical TGFβ signaling causes the transcription of HMGA2, which induces the transcription factors SNAIL and SLUG to repress the transcription of E-cadherin. Repressed E-cadherin allows for cells to undergo epithelial-to-mesenchymal transition (EMT), a key component of tumor metastasis and progression. Among immune cells, TGFβ modulates immune cell differentiation and function. TGFβ signaling promotes regulatory T cells by increasing expression of FOXP3. Concurrently, TGFβ suppresses the proliferation of CD8+ effector T cells and TH cells. TGFβ signaling also inhibits the cytotoxic effects of existing immune cells by reducing IFNγ production in NK cells, IFNα and TNF production in dendritic cells, and IFNγ, perforin, and granzyme B production in cytotoxic T cells. Overall, TGFβ promotes cancer progression and immunosuppression in the tumor microenvironment.

TGFβ exists initially as a complex bound to latency associated peptide (LAP) and latent TGFβ binding protein (LTBP). This complex, called the large latent complex (LLC), is released from the extracellular matrix. TGFβ is cleaved from the LLC by matrix metalloproteinases and integrins, leaving active dimeric TGFβ. Activated extracellular TGFβ binds to its membrane-bound heterotetrameric receptor complex, which includes TBRI and TβRII. While other ligands of the TGFβ superfamily have varied affinity for TBRI or TβRII, TGFβ itself binds only to TβRII. TGFβ binding to TβRII recruits TBRI and initiates a series of downstream signaling events via canonical and non-canonical signaling pathways. Canonical TGFβ signaling occurs via the SMAD pathway. Non-canonical TGFβ signaling encompasses a broad scope of additional pathways. After TGFβ binding, TβRII and phosphorylated TBRI can interact with several kinase signaling pathways including p38 MAPK, JNK, Rho/Rac, PI3K/Akt, ERK, and NF-κB. Crosstalk between TGFβ signaling pathways and these other pathways modulates signaling cascades to produce context-specific outcomes.

TGFβ type II receptor (TβRII): Transforming growth factor beta receptor 2 is a transmembrane receptor that binds TGFβ. Also known as TGFBR2. See, NCBI gene ID: 7048 (ncbi.nlm.nih.gov/gene/7048).

Transformed: As used herein, the term transformation encompasses all techniques by which a nucleic acid molecule might be introduced into a cell, including (but not limited to) transduction or transfection with viral vectors or plasmid vectors, or the introduction of DNA by electroporation, lipofection, or particle gun acceleration.

Treating or ameliorating a disease: Refers to a therapeutic intervention that decreases or inhibits a sign or symptom of a disease or pathological condition, such as a reduction in tumor size or tumor burden.

Vector: A nucleic acid molecule that can be introduced into a host cell (for example, by transfection or transduction), thereby producing a transformed host cell. Recombinant DNA vectors are vectors having recombinant DNA.

A vector can include nucleic acid sequences that permit it to replicate in a host cell, such as an origin of replication. A vector can also include one or more selectable marker genes and other genetic elements. An integrating vector is capable of integrating itself into a host nucleic acid (e.g., into a host chromosome). An expression vector is a vector that contains the necessary regulatory sequences to allow transcription and translation of an encoded gene or genes in a host cell.

One type of vector is a “plasmid,” which refers to a circular double-stranded DNA loop into which additional DNA segments can be inserted, such as by standard molecular cloning techniques. Another type of vector is a viral vector, wherein viral-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. A replication deficient viral vector is a vector that requires complementation of one or more regions of the viral genome required for replication due to a deficiency in at least one replication-essential gene function.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and, thereby, are replicated along with the host genome.

### Overview

The impact of TGFβ signaling on tumorigenesis has long been studied. Emphasis has been placed on the stromal effects of TGFβ, including promotion of epithelial-to-mesenchymal transition, invasion, fibrosis, and metastasis. However, TGFβ signaling also plays a dominant role in inhibiting the immune response by impeding the activity of infiltrating immune cells, influencing T-cell differentiation, and recruiting immunosuppressive cells to the tumor microenvironment. TGFβ inhibits dendritic cell activity by decreasing production of interferon alpha (IFNα) and tumor necrosis factor (TNF). Similarly, TGFβ signaling inhibits effector T cells by transcriptional regulation of granzyme B, perforin, and interferon in CD8⁺ T cells, demonstrating a direct negative impact on anti-tumor immunity. TGFβ blockade also enhances the effect of chemoradiation in murine models of colorectal cancer by direct effect on CD8⁺ T cells. This may occur via regulation of chemokine receptor CXCR3 expression, limiting migration to the tumor and altering the T cell receptor (TCR) signaling threshold. Importantly, this finding translates to clinical results. In patients with locally advanced rectal cancer, chemoradiation combined with TGFβ type I receptor (TBRI) inhibition resulted in more than double the historical response rate to chemoradiation alone, validating the importance of targeting TGFβ signaling as a therapeutic strategy. However, while this Phase II study generated positive results, many other TGFβ targeting agents including bispecific M7824 have failed to demonstrate benefit in large, randomized trials. Mixed results of TGFβ targeting agents may be attributable to multiple factors including patient selection, as TGFβ blockade may be most effective when TGFβ levels in the tumor are high. Consistent with this hypothesis, response rates in the Phase II study inversely correlated with the level of TGFβ intracellular mediator pSmad2 following administration of a TGFβ inhibitor.

In many tumor types, including gastrointestinal cancers and head and neck cancers, loss of TGFβ signaling components in the tumor epithelium results in feedback upregulation of TGFβ production resulting in elevated stromal TGFβ. Moreover, following radiation or certain chemotherapies, TGFβ is further elevated. Because of its role in cancer progression, immune suppression, and resistance to therapy, elevated extracellular TGFβ in the tumor microenvironment is correlated with worse disease outcomes. Extracellular TGFβ is therefore a valuable parameter for clinicians to consider as they evaluate potential therapeutic strategies. Currently, there are no available non-invasive methods to measure TGFβ levels in tumors. Available biopsy techniques are not only invasive but are not feasible for some patients depending on the location of the tumor. In the setting of metastatic disease, TGFβ levels may vary between individual tumors. Particularly in these patients, the availability of a non-invasive method for TGFβ detection and quantification would fulfill an unmet clinical need.

Positron Emission Tomography (PET) is an imaging modality with the potential to meet this need if an appropriate selective TGFβ radiotracer can be developed. To meet this need, disclosed herein are recombinant proteins, recombinant molecules, and compositions that specifically bind TGFβ and are suitable for use as radiotracers in PET imaging.

### Recombinant Proteins

Provided herein is a recombinant protein including (i) a transforming growth factor beta (TGFβ) binding domain and (ii) an antibody Fc fragment. The TGFβ-binding domain comprises or consists of a portion of TGFβ type II receptor (TβRII) that specifically binds TGFβ. In some examples, the TGFβ-binding domain consists of a portion of TβRII. In some examples, the TβRII is mammalian in origin, for example, a TβRII from mouse or human. In some examples, the portion of TβRII comprises an extracellular domain of TβRII. In some examples, the portion of TβRII consists of an extracellular domain of TβRII. In some examples, the recombinant protein can include a signal peptide, for example, to enhance or facilitate secretion of the recombinant protein when expressed by a host cell. However, in other examples, the signal peptide is cleaved (for example, upon secretion), and the protein does not include the signal peptide (referred to in some examples as the “mature” form of the protein). In several examples, the recombinant protein does not include a signal peptide.

In some examples, the portion of TβRII comprises an amino acid sequence having at least 80% sequence identity to SEQ ID NO: 3 or SEQ ID NO: 13 and specifically binds TGFβ; for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 3 or SEQ ID NO: 13 and specifically binds TGFβ. In a non-limiting example, the portion of TβRII comprises an amino acid sequence having at least 90% sequence identity to SEQ ID NO: 3 or SEQ ID NO: 13 and specifically binds TGFβ. In another non-limiting example, the portion of TβRII comprises an amino acid sequence having at least 95% sequence identity to SEQ ID NO: 3 or SEQ ID NO: 13 and specifically binds TGFβ. In a further non-limiting example, the portion of TβRII comprises SEQ ID NO: 3 or SEQ ID NO: 13. In another non-limiting example, the portion of TβRII consists of SEQ ID NO: 3 or SEQ ID NO: 13.

In some examples, the portion of TβRII is encoded by a nucleic acid sequence having at least 80% sequence identity to SEQ ID NO: 6 or SEQ ID NO: 14, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 6 or SEQ ID NO: 14. In a non-limiting example, the portion of TβRII is encoded by a nucleic acid sequence having at least 90% sequence identity to SEQ ID NO: 6 or SEQ ID NO: 14. In another non-limiting example, the portion of TβRII is encoded by a nucleic acid sequence having at least 95% sequence identity to SEQ ID NO: 6 or SEQ ID NO: 14. In a further non-limiting example, the portion of TβRII is encoded by a nucleic acid sequence including SEQ ID NO: 6 or SEQ ID NO: 14. In another non-limiting example, the portion of TβRII is encoded by a sequence consisting of SEQ ID NO: 6 or SEQ ID NO: 14.

The antibody Fc fragment is a sequence derived from the Fc region of an antibody. The antibody Fc fragment can be derived from any suitable antibody. Exemplary antibody classes include IgM, IgD, IgG, IgA and IgE. In some examples, the antibody Fc fragment is mammalian in origin, such as mouse or human antibody Fc fragment. The antibody Fc fragment can also be a chimera, such as a chimera of sequences derived from two or more origins (including artificial sequences). In some examples, the antibody Fc fragment is an IgG antibody fragment. In a non-limiting example, the antibody fragment is an IgG2 Fc fragment, for example, a human IgG2 Fc fragment. In some examples, the antibody Fc fragment includes a hinge region, a CH2 domain, and/or a CH3 domain. In some examples, the antibody Fc fragment includes at least two of: a hinge region, a CH2 domain, and a CH3 domain. In some examples, the antibody Fc fragment includes a hinge region, a CH2 domain, and a CH3 domain. In some examples, the hinge region, CH2 domain, and/or CH3 domain have the same origin as the antibody Fc fragment (e.g., an IgG2 antibody Fc fragment including a hinge region, a CH2 domain, and/or a CH3 domain also from IgG2). In other examples (e.g., an antibody Fc fragment chimera), the hinge region, CH2 domain, and/or CH3 domain have a different origin (e.g., an IgG2 antibody Fc fragment including an artificial hinge region, CH2 domain, and/or CH3 domain (or from a different class or allotype of an antibody)).

In some examples, the antibody Fc fragment comprises an amino acid sequence having at least 80% sequence identity to SEQ ID NO: 5, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 5. In a non-limiting example, the antibody Fe fragment comprises an amino acid sequence having at least 90% sequence identity to SEQ ID NO: 5. In a non-limiting example, the antibody Fc fragment comprises an amino acid sequence having at least 95% sequence identity to SEQ ID NO: 5. In another non-limiting example, the antibody Fc fragment comprises SEQ ID NO: 5. In a further non-limiting example, the antibody Fc fragment consists of SEQ ID NO: 5.

In some examples, the antibody Fc fragment is encoded by a nucleic acid sequence having at least 80% sequence identity to SEQ ID NO: 8, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 8. In a non-limiting example, the antibody Fc fragment is encoded by a nucleic acid sequence having at least 90% sequence identity to SEQ ID NO: 8. In another non-limiting example, the antibody Fc fragment is encoded by a nucleic acid having at least 95% sequence identity to SEQ ID NO: 8. In a further non-limiting example, the antibody Fc fragment is encoded by a nucleic acid sequence including SEQ ID NO: 8. In another non-limiting example, the antibody Fc fragment is encoded by a nucleic acid sequence consisting of SEQ ID NO: 8.

In some examples, the recombinant protein further includes a spacer sequence, for example, between the TGFβ binding domain and the antibody Fe fragment. In contrast to linker sequences, which are typically longer and lead to translation of flexible amino acids for more movement of a protein affixed to the Fc region, the spacer as described herein is only nucleotides necessary to maintain proper reading frame and/or facilitate cloning (e.g., restriction sites). In some examples, a recombinant protein disclosed herein does not include a linker sequence. In some examples, the spacer is less than 9 amino acids, for example, less than 8, less than 7, less than 6, less than 5, less than 4, less than 3, or less than 2 amino acids long. In some examples, the spacer is no more than 8 amino acids long. In some examples, the spacer is no more than 6 amino acids long. In some examples, the spacer is no more than 2 amino acids long. In some examples, the spacer is 0 to 10 amino acids, for example, 0 to 9, 0 to 8, 0 to 7, 0 to 6, 0 to 5, 0 to 4, 0 to 3, 0 to 2, 0 to 1, 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 4, 4 to 10, 4 to 9, 4 to 8, 4 to 7, 4 to 6, 4 to 5, 5 to 10, 5 to 8, or 5 to 6 amino acids. In some examples, the spacer is 0 to 1 amino acids. In some examples, the spacer is 0 to 8 amino acids. In some examples, the spacer is 1 to 8 amino acids. In some examples, the spacer is less than 25 nucleic acids, for example, less than 24, less than 22, less than 19, less than 18, less than 16, less than 13, less than 10, less than 7, less than 4, or less than 3 nucleic acids long. In some examples, the spacer is no more than 24 nucleic acids long. In some examples, the spacer is no more than 18 nucleic acids long. In some examples, the spacer is no more than 12 nucleic acids long. In some examples, the spacer is no more than 2 nucleic acids long. In some examples, the spacer is 1 to 25 nucleic acids, for example, 1 to 24, 1 to 23, 1 to 20, 1 to 17, 1 to 14, 1 to 11, 1 to 8, 1 to 5, 1 to 2, 2 to 24, 2 to 23, 2 to 20, 2 to 17, 2 to 14, 2 to 11, 2 to 8, 2 to 5, 3 to 23, 3 to 20, 3 to 17, 3 to 14, 3 to 11, 3 to 8, 3 to 5, 3 to 2, 4 to 23, 4 to 20, 4 to 17, 4 to 14, 4 to 11, 4 to 8, 4 to 5, 5 to 23, 5 to 20, 5 to 17, 5 to 14, 5 to 11, 5 to 8, 6 to 23, 6 to 20, 6 to 17, 6 to 14, 6 to 11, 8 to 23, 8 to 20, 8 to 17, 8 to 14, 8 to 11, 11 to 23, 11 to 20, 11 to 17, or 11 to 14 nucleic acids long. In some examples, the spacer is 1 to 24 nucleic acids. In some examples, the spacer is 2 to 24 nucleic acids. In some examples, a recombinant protein disclosed herein does not include a spacer sequence.

The spacer can include residues to maintain a proper reading frame such that when the recombinant protein is expressed, the TGFβ binding domain and the antibody Fc fragment are in the same reading frame. The spacer can include sequences that facilitate cloning (e.g., contain restriction enzyme sites). In some examples, the spacer comprises an amino acid sequence having at least 80% sequence identity to SEQ ID NO: 4, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 4. In a non-limiting example, the spacer comprises an amino acid sequence having at least 90% sequence identity to SEQ ID NO: 4. In a non-limiting example, the spacer comprises an amino acid sequence having at least 95% sequence identity to SEQ ID NO: 4. In another non-limiting example, the spacer includes SEQ ID NO: 4. In a further non-limiting example, the spacer consists of SEQ ID NO: 4. In some examples, the spacer is encoded by a nucleic acid sequence having at least 80% sequence identity to SEQ ID NO: 7, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 7. In a non-limiting example, the spacer is encoded by a nucleic acid sequence having at least 90% sequence identity to SEQ ID NO: 7. In another non-limiting example, the spacer is encoded by a nucleic acid having at least 95% sequence identity to SEQ ID NO: 7. In a further non-limiting example, the spacer is encoded by a nucleic acid sequence including SEQ ID NO: 7. In another non-limiting example, the spacer is encoded by a nucleic acid sequence consisting of SEQ ID NO: 7.

In some implementations, the recombinant protein disclosed herein includes a portion of TGFβ type II receptor (TβRII) and an IgG2 Fc domain. In some examples, the portion of the TGFβ type II receptor (TβRII) includes or consists of SEQ ID NO: 3 or SEQ ID NO: 13, and the IgG2 Fc domain includes or consists of SEQ ID NO: 5. In some examples, the recombinant protein further includes a signal peptide and/or spacer sequence between the portion of TβRII and the IgG2 Fc domain. In some examples, the protein does not include a signal peptide and/or a spacer sequence. In some examples, the recombinant protein includes a signal peptide and/or a restriction enzyme recognition site between the portion of TβRII and the IgG2 Fc domain.

In some examples, the recombinant protein disclosed herein includes an amino acid sequence having at least 80% sequence identity to SEQ ID NO: 1 or SEQ ID NO: 10, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 1 or SEQ ID NO: 10. In a non-limiting example, the recombinant protein disclosed herein includes an amino acid sequence having at least 90% sequence identity to SEQ ID NO: 1 or SEQ ID NO: 10. In a non-limiting example, the recombinant protein disclosed herein includes an amino acid sequence having at least 95% sequence identity to SEQ ID NO: 1 or SEQ ID NO: 10. In another non-limiting example, recombinant protein disclosed herein includes SEQ ID NO: 1 or SEQ ID NO: 10. In a further non-limiting example, the recombinant protein disclosed herein consists of SEQ ID NO: 1 or SEQ ID NO: 10.

In some examples, the recombinant protein disclosed herein is encoded by a nucleic acid sequence having at least 80% sequence identity to SEQ ID NO: 2 or SEQ ID NO: 11, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 2 or SEQ ID NO: 11. In a non-limiting example, the recombinant protein disclosed herein is encoded by a nucleic acid sequence having at least 90% sequence identity to SEQ ID NO: 2 or SEQ ID NO: 11. In another non-limiting example, the recombinant protein disclosed herein is encoded by a nucleic acid having at least 95% sequence identity to SEQ ID NO: 2 or SEQ ID NO: 11. In a further non-limiting example, the recombinant protein disclosed herein is encoded by a nucleic acid sequence including SEQ ID NO: 2 or SEQ ID NO: 11. In another non-limiting example, the recombinant protein disclosed herein is encoded by a nucleic acid sequence consisting of SEQ ID NO: 2 or SEQ ID NO: 11.

Variants (amino acid sequence variants) of the recombinant protein disclosed herein are also provided (e.g., variants of SEQ ID NO: 1 or SEQ ID NO: 10). Variants may be generated to improve binding affinity and/or other biological properties (e.g., optimize immunogenicity or half-life) of the recombinant protein. Amino acid sequence variants can be prepared by introducing modifications into the nucleotide sequence encoding the recombinant protein, or by peptide synthesis. Such modifications include, for example, deletions, insertions, and/or substitutions, of residues within the amino acid sequence. Any combination of deletion, insertion, and substitution can be made to arrive at the final construct, provided that the final construct retains TGFβ-binding.

In some examples, a variant is a conservative variant, which only includes conservative amino acid substitutions. Conservative amino acid substitutions are substitutions of an amino acid with a chemically similar amino acid, and thus the substitution is not expected to substantially affect or decrease a function of a protein, such as the ability of the protein to interact with a target (e.g., TGFβ). In some implementations, the recombinant protein disclosed herein comprises up to 40 (such as up to 5, up to 10, up to 15, up to 20, up to 25, up to 30, up to 35, or up to 40) amino acid substitutions (such as conservative amino acid substitutions) compared to the amino acid sequence set forth as one of SEQ ID NO: 1 or SEQ ID NO: 10. In some examples, the recombinant protein disclosed herein comprises up to 25 conservative amino acid substitutions as compared to SEQ ID NO: 1 or SEQ ID NO: 10. In some examples, the recombinant protein disclosed herein comprises up to 10 conservative amino acid substitutions as compared to SEQ ID NO: 1 or SEQ ID NO: 10.

The recombinant protein disclosed herein can, in some examples, include one or more synthetic amino acids in place of one or more naturally occurring amino acids. Such synthetic amino acids include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, oc-aminocycloheptane carboxylic acid, -(2-amino-2-norbornane)-carboxylic acid, γ-diaminobutyric acid, α,β-diaminopropionic acid, homophenylalanine, and α-tert-butylglycine.

The recombinant protein may also be glycosylated (e.g., N-glycosylated or O-glycosylated), amidated, carboxylated, phosphorylated, esterified, N-acylated, dimerized or polymerized, and/or conjugated. In some examples, the recombinant protein disclosed herein is N-glycosylated and/or O-glycosylated. In some examples, the average number of glycosylation moieties per recombinant protein molecule is at least 1, for example, at least 2, at least 3, at least 4, at least 5, or more. In a non-limiting example, the average number of glycosylation moieties per recombinant protein molecule is at least 2. In some examples, the average number of glycosylation moieties per recombinant protein molecule is 1 to 5, for example, 1 to 4, 1 to 3, 1 to 2, 2 to 5, 2 to 4, 2 to 3, 3 to 5, 3 to 4, or 4 to 5. In a non-limiting example, the average number of glycosylation moieties per recombinant protein molecule is 2 to 4. In some examples, the average number of glycosylation moieties per recombinant protein molecule is about 1, about 2, about 3, about 4, or about 5. In a non-limiting example, the average number of glycosylation moieties per recombinant protein molecule is about 3. In some examples, the recombinant protein disclosed herein is fucosylated. Expression of the recombinant protein disclosed herein in human cells, such as HEK 293 or HEK 293T cells, will generate desired post-translational modifications (e.g., glycosylation). Mammalian expression systems, such as CHO or NS0 cells, also generate human or human-like post-transcriptional modifications to expressed protein.

Also provided are recombinant molecules, which include a recombinant protein disclosed herein and a covalently attached metal ion chelator moiety. In some examples, the recombinant protein covalently attached metal ion chelator moiety does not include a signal peptide. The metal ion chelator moiety is a chemical compound that reacts with metal ions (such as copper or zirconium) to form stable, water-soluble metal complexes. Suitable chelators can be selected from a person of ordinary skill in the art. In some examples, the chelator is a copper chelator, for example, 1,4,7-triazacyclononane-1,4,7-triacetic acid (NOTA), 1,4,7,10-tetraazacyclododecane-1,4,7,10-tetraacetic acid (DOTA), or 1,4,7-triazacyclononane, 1-glutaric acid-4,7-acetic acid (NODAGA), DOTAGA, and CB-TE derivatives (e.g., MANOTA, see, Moreau et al. Dalton Trans. 46 (42): 14659-68, (2017)). In a non-limiting example, the chelator is 1,4,7-triazacyclononane-1,4,7-triacetic acid (NOTA). In some examples, the chelator is a zirconium chelator, for example desferrioxamine (DFO) or a DFO derivative. In some examples, the metal ion chelator is not a zirconium chelator.

In some examples, the chelator is a yttrium (e.g., yttrium-86) chelator, for example, EDTA derivatives or DOTA. In some examples, the chelator is a gallium (e.g., gallium-68) chelator, for example, NOTA or DOTA. In some examples, the chelator is a scandium (e.g., scandium-44) chelator, for example, DTPA (diethylenetriaminepentaacetic acid). In some examples, the chelator is a manganese (e.g., manganese-52) chelator, for example, DOTA.

The metal ion chelator moiety is covalently attached to the recombinant protein using known chemical reactions. In some examples, the metal ion chelator moiety is covalently attached using a p-SCN-Bn group, which reacts with exposed lysines. Other methods include, but are not limited to, modification of cysteine residues with maleimide conjugation, copper-catalyzed click chemistry, and enzyme-mediated conjugation. Further methods are described, for example, in Wei et al. (2020) ImmunoPET: Concept, Design, and Applications. Chemical Reviews (Vol. 120, Issue 8, pp. 3787-3851) American Chemical Society).

In some examples, at least 1 metal ion chelator moiety is covalently attached to the recombinant protein disclosed herein, such as at least 2, at least 3, at least 4, at least 5, at least 6, at least 7, at least 8, at least 9, at least 10, or more metal ion chelator moieties. In some examples, 1 to 10 metal ion chelator moieties are covalently attached to the recombinant protein disclosed herein, for example, 1 to 9, 1 to 8, 1 to 7, 1 to 6, 1 to 5, 1 to 4, 1 to 3, 1 to 2, 2 to 10, 2 to 9, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 9 to 10 metal ion chelator moieties are covalently attached to the recombinant protein disclosed herein. In some examples, 1 to 6 metal ion chelator moieties are covalently attached to the recombinant protein disclosed herein. In some examples, 3 to 7 metal ion chelator moieties are covalently attached to the recombinant protein disclosed herein. In some examples, 5 to 10 metal ion chelator moieties are covalently attached to the recombinant protein disclosed herein.

In some implementations, the recombinant molecule disclosed herein includes (i) a recombinant protein disclosed herein (including a portion of TGFβ type II receptor (TβRII) that specifically binds TGFβ and an IgG2 Fc domain), and (ii) a covalently attached copper chelator moiety. In some examples, the copper chelator moiety is a 1,4,7-triazacyclononane-1,4,7-triacetic acid (NOTA) moiety. In some examples, the recombinant molecule disclosed herein includes a portion of TGFβ type II receptor (TβRII) including or consisting of SEQ ID NO: 3 or SEQ ID NO: 13, an IgG2 Fc domain including or consisting of SEQ ID NO: 5, and a covalently attached 1,4,7-triazacyclononane-1,4,7-triacetic acid (NOTA) moiety.

In some examples, the recombinant molecule disclosed herein includes an amino acid sequence having at least 80% sequence identity to SEQ ID NO: 1 or SEQ ID NO: 10, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 1 or SEQ ID NO: 10 and a covalently attached 1,4,7-triazacyclononane-1,4,7-triacetic acid (NOTA) moiety. In a non-limiting example, the recombinant molecule disclosed herein includes an amino acid sequence having at least 90% sequence identity to SEQ ID NO: 1 or SEQ ID NO: 10 and a covalently attached 1,4,7-triazacyclononane-1,4,7-triacetic acid (NOTA) moiety. In a non-limiting example, the recombinant molecule disclosed herein includes an amino acid sequence having at least 95% sequence identity to SEQ ID NO: 1 or SEQ ID NO: 10 and a covalently attached 1,4,7-triazacyclononane-1,4,7-triacetic acid (NOTA) moiety. In another non-limiting example, the recombinant molecule disclosed herein includes SEQ ID NO: 1 or SEQ ID NO: 10 and a covalently attached 1,4,7-triazacyclononane-1,4,7-triacetic acid (NOTA) moiety. In a further non-limiting example, the recombinant molecule disclosed herein consists of SEQ ID NO: 1 or SEQ ID NO: 10 and a covalently attached 1,4,7-triazacyclononane-1,4,7-triacetic acid (NOTA) moiety.

The recombinant protein and recombinant molecule disclosed herein specifically bind TGFβ. Assays to determine specific binding are generally known, for example, competitive binding assays such as ELISA or surface plasmon resonance. The K_{D} (binding affinity) can be determined using such assays.

The recombinant protein or recombinant molecule disclosed herein can be derivatized or labeled (e.g., linked to another molecule or peptide). In general, the recombinant protein or recombinant molecule disclosed herein is derivatized such that the binding to TGFβ is not abolished or adversely affected by the derivatization or labeling. For example, the recombinant protein or recombinant molecule can be functionally linked (by chemical coupling, genetic fusion, noncovalent association or otherwise) to one or more molecular entities, such as a detectable marker, an effector molecule, or other protein or peptide, and retain binding affinity for TGFβ.

In some implementations, the recombinant protein or recombinant molecule disclosed herein does not include a linker between the TGFβ binding domain and the antibody Fc domain. In some implementations, the recombinant protein or recombinant molecule disclosed herein does not include a fragment antigen binding (Fab) region of an antibody. In some implementations, the recombinant protein or recombinant molecule disclosed herein does not include a sequence or domain of a TGFβ receptor other than TβRII, for example, does not include a sequence or domain from TGFβ type I receptor (TβRI) or TGFβ type III receptor (TβRIII).

### Nucleic Acids, Vectors, and Protein Expression

Nucleic acid molecules encoding a recombinant protein disclosed herein are provided. In some examples, the nucleic acid molecule includes a sequence encoding a protein having at least 80% sequence identity to SEQ ID NO: 1 or SEQ ID NO: 10, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 1 or SEQ ID NO: 10. In some examples, the nucleic acid molecule includes a sequence encoding a protein having at least 95% sequence identity to SEQ ID NO: 1 or SEQ ID NO: 10. In some examples, the nucleic acid molecule includes a sequence encoding a protein having at least 98% sequence identity to SEQ ID NO: 1 or SEQ ID NO: 10. In some examples, the nucleic acid molecule includes or consists of a sequence encoding SEQ ID NO: 1 or SEQ ID NO: 10.

In some examples, the nucleic acid molecule encoding a recombinant protein disclosed herein has at least 80% sequence identity to SEQ ID NO: 2 or SEQ ID NO: 11, for example, at least 85%, at least 87%, at least 90%, at least 91%, at least 92%, at least 93%, at least 94%, at least 95%, at least 96%, at least 97%, at least 98%, at least 99%, or 100% sequence identity to SEQ ID NO: 2 or SEQ ID NO: 11. In a non-limiting example, the nucleic acid molecule encoding a recombinant protein disclosed herein has at least 90% sequence identity to SEQ ID NO: 2 or SEQ ID NO: 11. In a further example, the nucleic acid molecule encoding a recombinant protein disclosed herein has at least 95% sequence identity to SEQ ID NO: 2 or SEQ ID NO: 11. In another example, the nucleic acid molecule encoding a recombinant protein disclosed herein includes SEQ ID NO: 2 or SEQ ID NO: 11. In another example, the nucleic acid molecule encoding a recombinant protein disclosed herein consists of SEQ ID NO: 2 or SEQ ID NO: 11.

In some implementations, a nucleic acid molecule encoding a disclosed recombinant protein is included in a vector (such as a plasmid or viral vector). In some examples, the vector is an expression vector that facilitates protein expression in a host cell (e.g. E. coli, Chinese hamster ovary (CHO) or embryonic human kidney (HEK293 or HEK 293T)). The expression vector can be a vector for transient or stable protein expression. In some examples, the vector includes a promoter (e.g., Elongation Factor-1α (EF-1α)) operably linked to a nucleic acid molecule encoding a recombinant protein disclosed herein (e.g., SEQ ID NO: 2 or SEQ ID NO: 11). The vector can include additional expression control sequences, such as one or more enhancers, transcription and/or translation terminators, and initiation sequences. In some examples, the vector includes one or more of: the R segment and part of the U5 sequence (R-U5′) of Human T-Cell Leukemia Virus Type 1 Long Terminal Repeat (for durable stable transgene expression in vivo), a Simian Virus 40 late polyadenylation signal (to enable high levels of steady-state mRNA), a minimal E. coli origin of replication fragment (to allow transcription in E. coli), and a EF1-HTLV promoter. In some examples, a nucleic acid encoding the recombinant protein disclosed herein is included in a plasmid vector (e.g., pFUSE-hIgG2-Fc1). Exemplary viral vectors include retrovirus (e.g., MoMLV or lentivirus), adenovirus, adeno-associated virus, vaccinia virus, and fowlpox vectors. In some examples, the vector includes a selectable marker (such as an antibiotic resistance gene, for example Zeocin™) and/or a reporter gene (such as green fluorescent protein (GFP)).

Also provided are host cells that express a recombinant protein disclosed herein, or contain a nucleic acid disclosed herein, or a vector including a nucleic acid disclosed herein. The host cell can be prokaryotic (e.g., bacterial cells) or eukaryotic (e.g., mammalian cells). In some examples, the host cell is a mammalian cell, such as CHO, HEK 293, or HEK 293T cells.

In some implementations, the host cell is transformed with a nucleic acid disclosed herein, or a vector including a nucleic acid disclosed herein. In a non-limiting example, the host cell is transformed with a nucleic acid sequence encoding SEQ ID NO: 1 or SEQ ID NO: 10; or is transformed with a vector including a nucleic acid sequence encoding SEQ ID NO: 1 or SEQ ID NO: 10. In another non-limiting example, the host cell is transformed with a nucleic acid sequence including or consisting of SEQ ID NO: 2 or SEQ ID NO: 11. In a further non-limiting example, the host cell is transformed with a vector including a nucleic acid sequence including or consisting of SEQ ID NO: 2 or SEQ ID NO: 11. Following transformation, cells containing the nucleic acid or vector can be selected and/or enriched, for example, by using antibiotic selection. In some examples, the cells are expanded, for example, by cell culture. In some examples, some or all of the transformed cells are cryopreserved for later use.

In some implementations, a host cell transformed with a nucleic acid disclosed herein, or a vector including a nucleic acid disclosed herein, expresses a recombinant protein disclosed herein. The recombinant protein can be purified from the transformed host cell, for example, by using a protein purification column (e.g., Protein A or Protein G Sepharose column), and a metal chelator group (e.g., NOTA) is covalently attached to the purified recombinant protein, thereby producing a recombinant molecule disclosed herein.

Protein expression by a host cell can be transient or stable. In a non-limiting example, HEK 293T cells are transformed with a nucleic acid sequence encoding Fc:TβRII (e.g., SEQ ID NO: 1 or SEQ ID NO: 10), or a vector including a nucleic acid sequence encoding Fc:TβRII, and the HEK 293T cells stably express the encoded Fc:TβRII protein.

### Compositions

Compositions including a recombinant molecule disclosed herein and a radionuclide (e.g., copper-64) are also provided. In some examples, the radionuclide is bound to the metal ion chelator (e.g., NOTA). Also provided are methods of producing the composition, which includes incubating a recombinant molecule disclosed herein with a radionuclide (e.g., copper-64).

A radionuclide is a radioactive nuclide (atom). In some implementations, the radionuclide is a metal radionuclide. Exemplary metal radionuclides include copper-64 (⁶⁴Cu), copper-61 (⁶¹Cu), copper-60 (⁶⁰Cu), zirconium-89 (⁸⁹Zr), gallium-66 (⁶⁶Ga), gallium-68 (⁶⁸Ga), yttrium-86 (⁸⁶Y), scandium-44 (⁴⁴Sc), manganese-52 (⁵²Mn), and technetium-94m (^{94m}Tc). In a non-limiting example, the radionuclide is copper-64 (⁶⁴Cu). In some examples, the radionuclide is not a zirconium radionuclide (e.g., is not zirconium-89).

Also provided herein are pharmaceutical compositions including a recombinant protein, recombinant molecule, nucleic acid, vector, or composition as disclosed herein, in a pharmaceutically acceptable carrier. The pharmaceutical composition will typically be a solution of an agent (e.g., recombinant protein, recombinant molecule, nucleic acid, vector, or composition as disclosed herein) dissolved in a pharmaceutically acceptable carrier, preferably an aqueous carrier (e.g., PBS or saline). A variety of aqueous carriers can be used, e.g., buffered saline and the like. These solutions are sterile and generally free of undesirable matter. Such compositions may be sterilized by conventional sterilization techniques. The compositions may contain pharmaceutically acceptable auxiliary substances as required to approximate physiological conditions, such as pH adjusting and buffering agents, toxicity adjusting agents and the like, for example, sodium acetate, sodium chloride, potassium chloride, calcium chloride, sodium lactate and the like. The concentration of an agent in these formulations can vary, and will be selected primarily based on fluid volumes, viscosities, body weight and the like in accordance with the particular mode of administration selected and the subject's needs. Actual methods for preparing pharmaceutical compositions will be known or apparent to a practitioner and are described in more detail in publications such as Remington's Pharmaceutical Sciences, 23rd Edition, Academic Press, Elsevier, (2020).

The pharmaceutical compositions can be formulated for any appropriate administration route (e.g., injection, such as subcutaneous, intramuscular, intradermal, intraperitoneal, or intravenous injection). In a non-limiting example, the pharmaceutical composition is formulated for intravenous administration, and includes about 1 mg to 1000 mg of an agent (e.g., the recombinant protein, recombinant molecule, or composition disclosed herein) per kilogram (kg) of the subject, for example, 1 to 900 mg, 1 to 800 mg, 1 to 700 mg, 1 to 600 mg, 1 to 500 mg, 1 to 400 mg, 1 to 300 mg, 1 to 200 mg, 1 to 100 mg, 1 to 50 mg, 10 to 1000 mg, 10 to 900 mg, 10 to 800 mg, 10 to 700 mg, 10 to 600 mg, 10 to 500 mg, 10 to 400 mg, 10 to 300 mg, 10 to 200 mg, 10 to 100 mg, 10 to 50 mg, 50 to 1000 mg, 50 to 900 mg, 50 mg to 800 mg, 50 mg to 700 mg, 50 mg to 600 mg, 50 mg to 500 mg, 50 mg to 400 mg, 50 mg, to 300 mg, 50 mg to 200 mg, 50 mg to 100 mg, 100 mg to 1000 mg, 100 mg to 900 mg, 100 mg to 800 mg, 100 mg to 700 mg, 100 mg to 600 mg, 100 mg to 500 mg, 100 mg to 400 mg, 100 mg to 300 mg, 100 mg to 200 mg, 200 mg to 1000 mg, 200 mg to 900 mg, 200 mg to 800 mg, 200 mg to 700 mg, 200 mg to 600 mg, 200 mg to 500 mg. 200 mg to 400 mg, 200 mg to 300 mg, 300 mg to 1000 mg, 400 mg to 1000 mg, 500 mg to 1000 mg, 600 mg to 1000 mg, 700 mg to 1000 mg, or 800 mg to 1000 mg per kilogram (kg) of the subject. In some examples, the pharmaceutical composition includes about 50 mg to 1000 mg of an agent per kilogram (kg) of the subject. In some examples, the pharmaceutical composition includes about 100 mg to 1000 mg of an agent per kilogram (kg) of the subject. In some examples, the pharmaceutical composition includes about 50 mg to 500 mg of an agent per kilogram (kg) of the subject. In some examples, the pharmaceutical composition includes about 100 mg to 500 mg of an agent per kilogram (kg) of the subject.

In some implementations, a dose is determined by the radioactivity of the pharmaceutical composition, for example, a single dose is 10 to 1000 megabecquerels (MBq) per kilogram (kg) of the subject. In some examples, a single dose is 100 to 600 megabecquerels (MBq) per kilogram (kg) of the subject. In some examples, a single dose is 100 to 400 megabecquerels (MBq) per kilogram (kg) of the subject. In some examples, a single dose is 200 to 600 megabecquerels (MBq) per kilogram (kg) of the subject. In some implementations, 50-200 microliters of the pharmaceutical agent is administered to a subject.

An appropriate dose can be determined by a skilled clinician based on factors such as the subject, treatment history, cancer type, tumor load and type, clinical stage and grade of the disease, overall health of the subject, and other factors. Single or multiple administrations of a composition or pharmaceutical composition can be administered to the subject.

### Methods of Detecting TGFβ

Also provided are methods of detecting transforming growth factor beta (TGFβ) in a subject, including administering a composition (including a pharmaceutical composition) disclosed herein, and detecting the composition (or pharmaceutical composition) in the subject, thereby detecting TGFβ in the subject. The subject is a living multi-cellular vertebrate. In some examples, the subject is mammalian, for example, a human, non-human primate, cat, dog, mouse, or rat. In a non-limiting example, the subject is human. In some examples, a subject having a disease or cancer associated with elevated TGFβ is selected for the detection of TGFβ. In some implementations, the subject has cancer. In some examples, the subject has or is suspected of having elevated TGFβ levels.

Administration of any composition (including pharmaceutical compositions) disclosed herein can be local or systemic. Exemplary routes of administration include, but are not limited to, oral, injection (such as subcutaneous, intramuscular, intradermal, intraperitoneal, intravenous), sublingual, rectal, transdermal (for example, topical), intranasal, vaginal, and inhalation routes. In some examples, a composition is injected or infused into a tumor, or close to a tumor (local administration), or administered to the peritoneal cavity. In some implementations, administration is by intravenous injection. Appropriate routes of administration can be determined by a skilled clinician based on factors such as the subject, the condition being treated, and other factors. Exemplary doses are provided herein in the “Compositions” section.

Detection includes any process for identifying the presence of the composition or pharmaceutical composition. In some examples, detection includes detection of radioactivity from a composition or pharmaceutical composition disclosed herein. In some examples, detection is achieved using positron emission tomography (PET). In some examples, detection is in vivo (e.g., a positron emission tomography scan of a subject). In some examples, TGFβ is detected using a disclosed composition (e.g., a pharmaceutical composition disclosed here) and positron emission tomography (PET). In some aspects, the composition or pharmaceutical composition provides a proxy signal for the presence of TGFβ (TGFβ is indirectly detected by its interaction with a disclosed composition, which is detectable). Detection can be quantitative or qualitative. In a non-limiting example, TGFβ is quantitatively detected in a subject by a PET scan.

When a composition or pharmaceutical composition disclosed herein is used for PET imaging, a dose is typically administered to a subject within a day of the imaging, for example, within 12 hours, within 6 hours, within 4 hours, within 2 hours, within 1 hour, or within 30 minutes of imaging the subject. In some examples, a dose is administered within 1 hour of imaging.

In some implementations, the subject has a tumor or cancer. In some implementations, a subject having cancer is selected for administration of the composition or pharmaceutical composition disclosed herein. In some examples, the cancer is a solid tumor or cancer, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma (such as an HPV-positive HNSCC), and lymphatic tumors (including B-cell and T-cell malignant lymphoma).

In some examples, the cancer is a liquid tumor or cancer, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

In some examples, the cancer is associated with elevated TGFβ. In a non-limiting example, the subject has a gastrointestinal cancer, head and neck cancer, breast cancer, or colorectal cancer.

### Kits

Also provided are kits. In some examples, the kit includes a recombinant protein, recombinant molecule, composition, or pharmaceutical composition disclosed herein. In other examples, the kit includes a nucleic acid encoding a recombinant protein disclosed herein, a vector including such nucleic acids, or a cell containing such nucleic acids or vectors. In some implementations, the kit further comprises host cells, media, an antibiotic, a transformation reagent, a nucleic acid purification column (e.g., silica column), a nucleic acid purification reagent (e.g, wash and/or column elution solution), a protein purification column (e.g., a size exclusion column (e.g., PD-10), binding column (e.g., Protein A or G Sepharose column), etc.), a protein purification reagent (e.g, wash and/or column elution solution), a buffer, a metal ion chelator (e.g., p-SCN-Bn-NOTA), a reagent for conjugating a metal ion chelator (e.g., a buffer), a pharmaceutically acceptable carrier, a radionuclide or radionuclide source (e.g., [⁶¹Cu]CuCl₂), and/or a radiolabeling reagent.

In one example, the kit includes a recombinant protein or a recombinant molecule disclosed herein, and further includes one or more of: a metal ion chelator (e.g., p-SCN-Bn-NOTA), a reagent for conjugating a metal ion chelator, a protein purification column, a protein purification reagent, a buffer, a pharmaceutically acceptable carrier, a radionuclide or radionuclide source (e.g., [⁶⁴Cu]CuCl₂), and a radiolabeling reagent. In another example, the kit includes a nucleic acid encoding a recombinant protein disclosed herein, a vector including such nucleic acids, and/or a cell containing such nucleic acids or vectors; and one or more of: host cells, media, a transformation reagent, a nucleic acid purification column, a nucleic acid purification reagent, a protein purification column, a protein purification reagent, a metal ion chelator (e.g., p-SCN-Bn-NOTA), a reagent for conjugating a metal ion chelator, a buffer, a pharmaceutically acceptable carrier, a radionuclide or radionuclide source (e.g., [⁶⁴Cu]CuCl₂), and a radiolabeling reagent.

In some examples, the kit components are provided in separate containers. Such kits are useful, for example, for practicing any of the methods disclosed herein. In some examples, the kit includes instructions for practicing a method disclosed herein.

### Additional Aspects

[Clause 1] A recombinant molecule comprising:
(i) a recombinant protein comprising (a) a transforming growth factor beta (TGFβ) binding domain consisting of a portion of TGFβ type II receptor (TβRII), wherein the portion specifically binds TGFβ, and (b) an antibody Fc fragment; and
(ii) a metal ion chelator moiety covalently attached to the recombinant protein.
   [Clause 2] The recombinant molecule of clause 1, wherein the portion of TβRII comprises an extracellular domain of TβRII.
   [Clause 3] The recombinant molecule of clause 1 or clause 2, wherein the portion of TβRII comprises an amino acid sequence having at least 90% sequence identity to SEQ ID NO: 3 or SEQ ID NO: 13.
   [Clause 4] The recombinant molecule of any one of clauses 1-3, wherein the portion of TβRII comprises or consists of SEQ ID NO: 3 or SEQ ID NO: 13.
   [Clause 5] The recombinant molecule of any one of clauses 1-4, wherein the antibody Fe fragment is an IgG2 Fc fragment.
   [Clause 6] The recombinant molecule of any one of clauses 1-5, wherein the antibody Fe fragment comprises a hinge region, CH2 domain, and/or CH3 domain.
   [Clause 7] The recombinant molecule of any one of clauses 1-6, wherein the antibody Fc fragment comprises an amino acid sequence having at least 90% sequence identity to SEQ ID NO: 5.
   [Clause 8] The recombinant molecule of any one of clauses 1-7, wherein the antibody Fc fragment comprises or consists of SEQ ID NO: 5.
   [Clause 9] The recombinant molecule of any one of clauses 1-8, wherein the metal ion chelator moiety is a copper chelator.
   [Clause 10] The recombinant molecule of any one of clauses 1-9, wherein the metal ion chelator moiety is 1,4,7-triazacyclononane-1,4,7-triacetic acid (NOTA), 1,4,7,10-tetraazacyclododecane-1,4,7,10-tetraacetic acid (DOTA), or 1,4,7-triazacyclononane, 1-glutaric acid-4,7-acetic acid (NODAGA).
   [Clause 11] The recombinant molecule of any one of clauses 1-10, wherein the recombinant protein does not comprise a linker between the TGFβ binding domain and the antibody Fc domain.
   [Clause 12] The recombinant molecule of any one of clauses 1-11, wherein the recombinant molecule does not comprise a fragment antigen binding (Fab) region of an antibody.
   [Clause 13] The recombinant molecule of any one of clauses 1-12, wherein the recombinant protein further comprises a spacer sequence of 8 or fewer amino acids between the portion of TβRII and the IgG2 Fc domain.
   [Clause 14] The recombinant molecule of clause 13, wherein the recombinant protein consists of in N-terminus to C-terminus order, the portion of TβRII, the spacer sequence of 8 or fewer amino acids, and the IgG2 Fc domain, and the metal ion chelator moiety is a NOTA moiety.
   [Clause 15] The recombinant molecule of any one of clauses 1-14, wherein the recombinant protein comprises an amino acid sequence having at least 90% sequence identity to SEQ ID NO: 1 or SEQ ID NO: 10.
   [Clause 16] The recombinant molecule of any one of clauses 1-15, wherein the recombinant protein comprises or consists of SEQ ID NO: 1 or SEQ ID NO: 10.
   [Clause 17] A composition comprising the recombinant molecule of any one of clauses 1-16 and a radionuclide bonded to the metal ion chelator.
   [Clause 18] The composition of clause 17, wherein the radionuclide is copper-64.
   [Clause 19] A nucleic acid molecule encoding the recombinant protein of any one of clauses 1-16.
   [Clause 20] The nucleic acid molecule of clause 19, comprising a nucleic acid sequence having at least 90% sequence identity to SEQ ID NO: 2 or SEQ ID NO: 11.
   [Clause 21] The nucleic acid molecule of clause 19 or 20, comprising or consisting of SEQ ID NO: 2 or SEQ ID NO: 11.
   [Clause 22] A vector comprising the nucleic acid molecule of any one of clauses 19-21.
   [Clause 23] A cell expressing the recombinant protein of any one of clauses 1-16, optionally wherein the recombinant protein comprises or consists of SEQ ID NO: 1 or SEQ ID NO: 10.
   [Clause 24] A cell comprising the nucleic acid of any one of clauses 19-21, or the vector of clause 22, optionally wherein the nucleic acid or vector encodes an amino acid sequence comprising or consisting of SEQ ID NO: 1 or SEQ ID NO: 10.
   [Clause 25] The cell of clause 23 or 24, wherein the cell is a mammalian cell.
   [Clause 26] The cell of clause 25, wherein the mammalian cell is a human cell.
   [Clause 27] A kit, comprising one or more of: the recombinant molecule of clauses 1-16, the composition of clause 17 or 18, the nucleic acid of clauses 19-21, the vector of clause 22, or the cell of clauses 23-26.
   [Clause 28] The kit of clause 27, further comprising a radionuclide.
   [Clause 29] A method of producing the recombinant molecule of any one of clauses 1-16, comprising expressing the recombinant protein in a mammalian cell and subsequently attaching the covalent metal ion chelator moiety.
   [Clause 30] The method of clause 29, wherein the mammalian cell is a human cell.
   [Clause 31] The method of clause 30, wherein the human cell is a HEK 293T cell.
   [Clause 32] A method of producing the composition of clause 17 or 18, comprising incubating the recombinant molecule of any one of clauses 1-16 with the radionuclide.
   [Clause 33] A method of detecting transforming growth factor beta (TGFβ) in a subject, comprising: (i) administering the composition of clauses 17 or 18 to the subject; and (ii) detecting the composition in the subject, thereby detecting TGFβ in the subject.
   [Clause 34] The method of clause 33, wherein detecting comprises detecting by positron emission tomography (PET).
   [Clause 35] The method of clause 33 or 34, wherein administering comprises intravenous injection.
   [Clause 36] The method of any one of clauses 33 to 35, wherein the subject has cancer.

### EXAMPLES

### Example 1

### Fc:TβRII PET Tracer Design

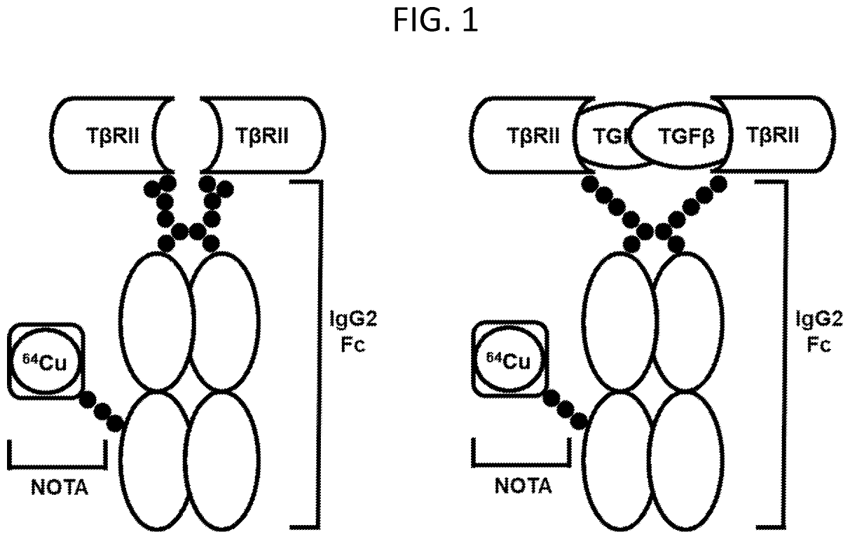

The Fc:TβRII PET radiotracer has four main components: the TGFβ-binding TβRII domain, the solubilizing Fc domain, the conjugated metal ion chelator, and the PET radionuclide. A general schematic is provided in FIG. 1. The rationales supporting each of these design elements are discussed below.

TGFβ-binding TβRII domain: TβRII is the native binding target of TGFβ, and selectively binds to TGFβ in vitro and in vivo.

Solubilizing Fc domain: The TβRII extracellular domain was solubilized by recombination with the human IgG2 Fc fragment, including its CH2 and CH3 domains and hinge region. IgG Fc fragments self-dimerize via hydrophobic contacts at the CH2 domains and link covalently via disulfide bonds in the hinge region. This dimerization is desirable for the proposed construct because TGFβ exists as a dimer (FIG. 1, right). The hinge regions of recombinant Fc fragments are sometimes modified or omitted to increase serum half-life and prolong a desired biological activity. In this case, a shorter biological half-life is desirable for more rapid clearance from preclinical and clinical subjects after PET imaging. As discussed above, antibody fragments (Fc or Fab, ˜50 kDa) show better tumor penetration and more rapid clearance from the body compared with full-length antibodies (˜150 kDa). The IgG2 subclass was selected for the proposed radiotracer because target cell depletion by ADCC or other effector functions was not necessarily desirable from an imaging standpoint. Human IgG2 was selected because the ultimate goal for the construct is clinical diagnostic use, and because any given mouse will not be dosed more than three times with the radiotracer, limiting an anti-human immune response. Notably, the Fc domain has the added logistical advantage of facilitating protein purification using protein A or protein G Sepharose.

Conjugated metal ion chelator: The recombinant protein is converted to the radiolabeling precursor by covalently appending the 1,4,7-triazacyclononane-1,4,7-triacetic acid (NOTA) copper chelator via a p-SCN-Bn reactive moiety. This molecule is known in the field of immunoPET for conjugating to proteins and chelating to copper under mild conditions, though alternatives are available.

PET radionuclide: ⁶⁴Cu is a positron-emitting radionuclide with a decay half-life of 12.7 hr, which is predicted to most closely match the biological half-life of an antibody fragment (in contrast to the most common immunoPET radionuclide, ⁸⁹Zr, which has a half-life of 78.4 hours). ⁶⁴Cu can be obtained commercially as [⁶⁴Cu]CuCl₂, from which the chloride can be displaced by the NOTA chelator under mildly acidic conditions.

### Example 2

### Expression of Fc:TβRII Construct

A plasmid vector containing the human IgG2 Fc and hinge region was obtained from InvivoGen (pFUSE-hIgG2-Fc1; Cat. Code pfuse-hfc1). This plasmid contains a fused Elongation Factor-1α (EF-1α) core promoter and the R segment and part of the U5 sequence (R-U5′) of the Human T-Cell Leukemia Virus Type 1 Long Terminal Repeat for durable stable transgene expression in vivo; a Simian Virus 40 late polyadenylation signal to enable high levels of steady-state mRNA; a minimal E. coli origin of replication fragment to allow transcription in E. coli; and a composite promoter combining the human cytomegalovirus immediate-early gene 1 enhancer and the core promoter of the human ferritin light chain gene to drive expression of the Zeocin™-resistance gene (Sh ble gene). This vector contains a multiple cloning site region between the EF1-HTLV promoter and the IgG2-Fc1 domain.

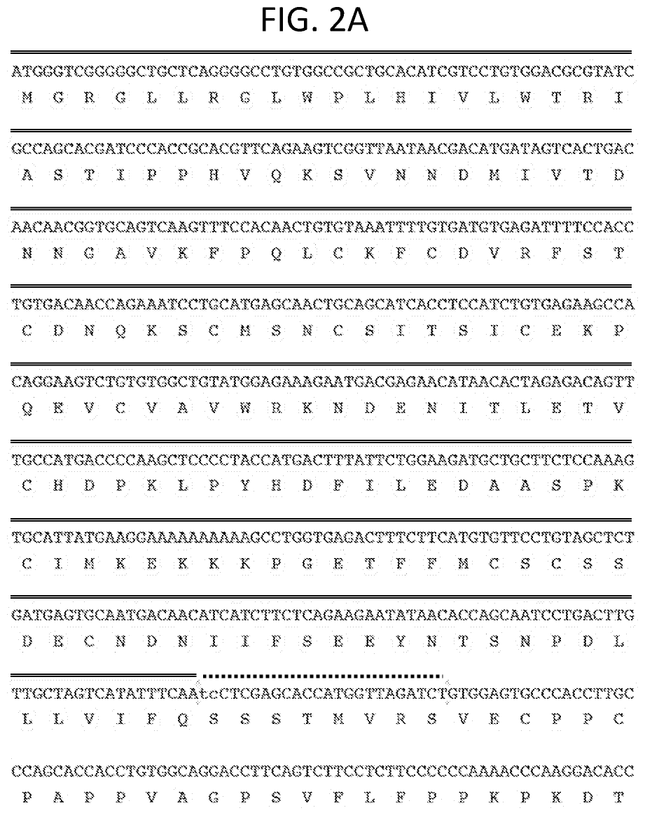

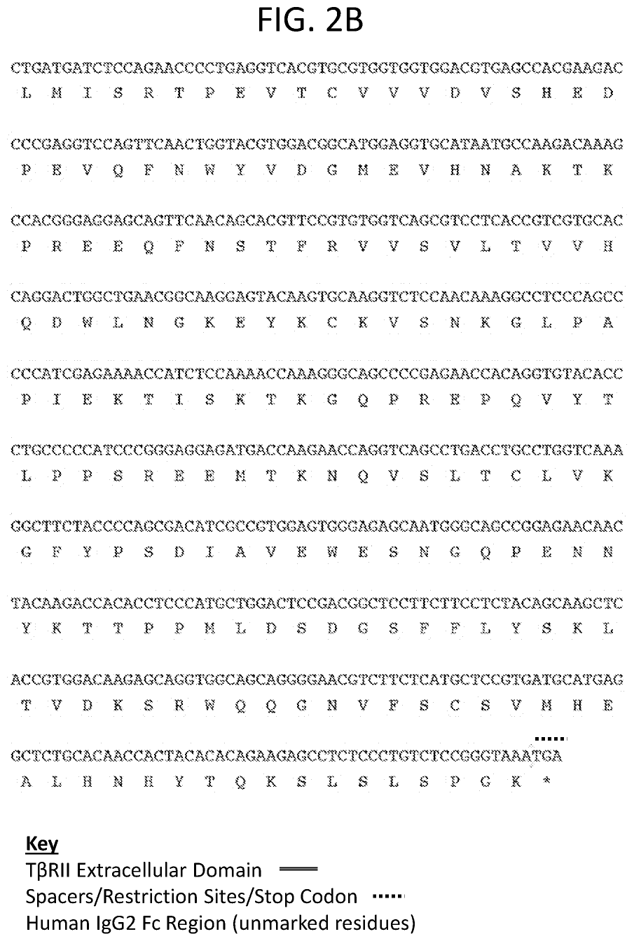

A fragment sequence encoding the extracellular domain of human TβRII (nucleotides 1-498; SEQ ID NO: 6) was obtained from Integrated DNA Technologies (IDT). An EcoRI restriction site was included at the 5′ end of the sequence and an XhoI restriction site was included at the 3′ end of the sequence. In addition, a 2 nucleotide spacer was included between the TβRII and XhoI on the 3′ end to keep the correct reading frame in line with IgG2. EcoRI and XhoI restriction enzymes were used to cleave the hTβRII gene fragment and the hIgG2 Fc-hinge plasmid. The cleaved fragments were ligated together to form Fc:TβRII (FIGS. 2A-2B).

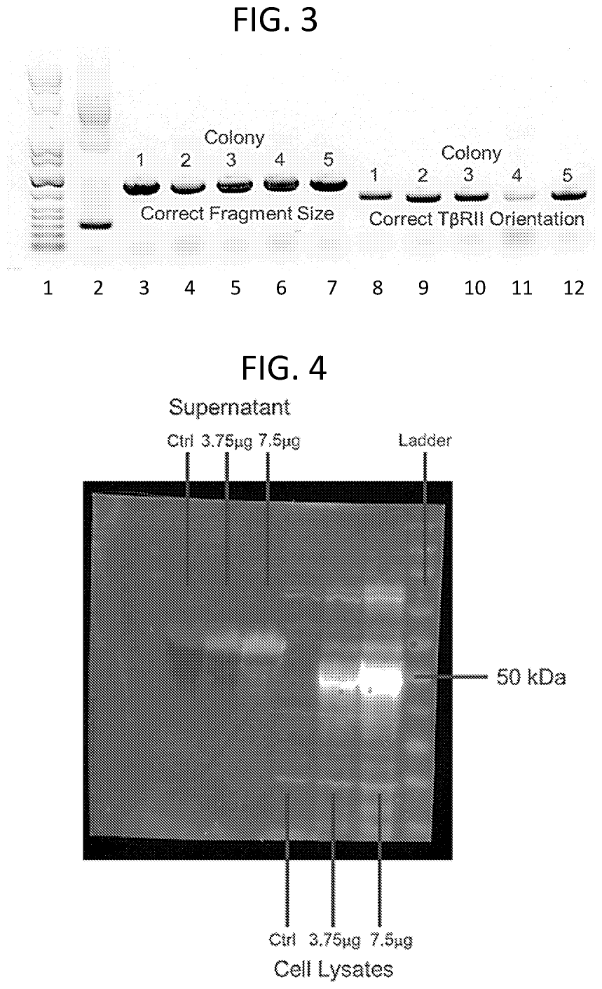

The ligated plasmid was transformed into E. coli and spread on Zeocin™ containing agar plates (Zeocin™ is a formulation of phleomycin D1 and is a selective antibiotic for the Sh ble gene; InvivoGen Cat. No: ant-zn-1p). Single colonies were selected and grown in LB plates supplemented with Zeocin™. Colony PCR was performed on candidate clones to confirm the presence of the insert in the pFUSE-hIgG2-Fc1 vector and correct orientation (FIG. 3). The protein was expressed in E. coli and purified. As expected for the glycosylated protein, the purified protein was 65 kDa (see, FIG. 4).

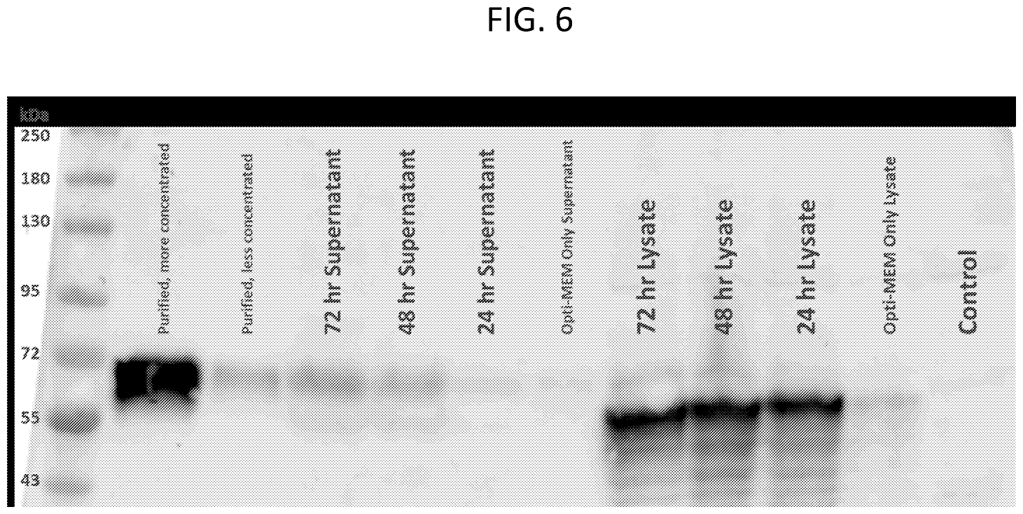

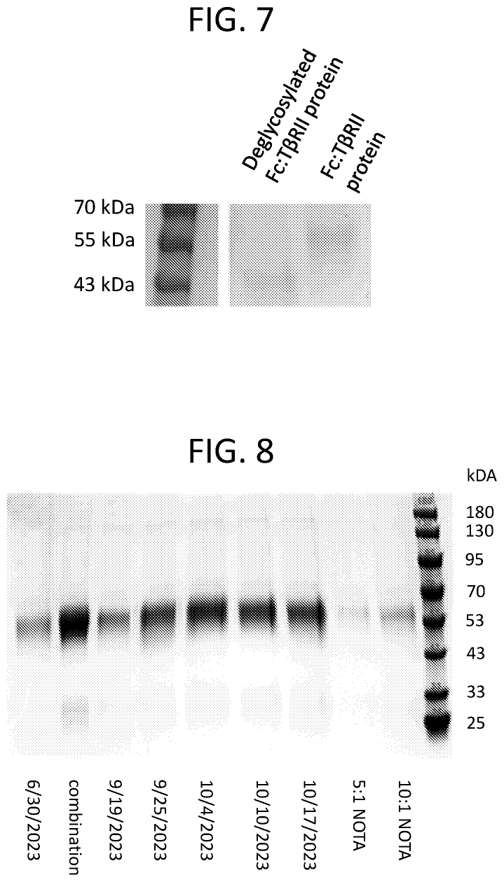

The complete Fc: TBRI plasmid was transfected into cultured HEK 293T cells (ATCC CRL-3216™) for protein expression using Lipofectamine™ 3000. Correct protein folding and size were verified by Western Blot using a TβRII antibody (see FIG. 6, a Western blot of Fc:TβRII protein in supernatant or cell lysate fractions at 24, 48, and 72 hours). A mammalian system was selected for protein expression to produce the desired Fc post-translational modifications (N-glycosylation) and to prevent the formation of inclusion bodies. To confirm that the Fc:TβRII protein was glycosylated when expressed by HEK 293T cells, isolated Fc:TβRII protein was de-glycosylated by a 3 hour incubation with N-glycanase using an Agilent Enzymatic Deglycosylation Kit (GK80110) and analyzed by Western blot. FIG. 7 shows that the deglycosylated protein was the expected 45 kDa, confirming that the 65 kDa Fc:TβRII protein expressed by HEK 293T cells runs larger due to glycosylation.

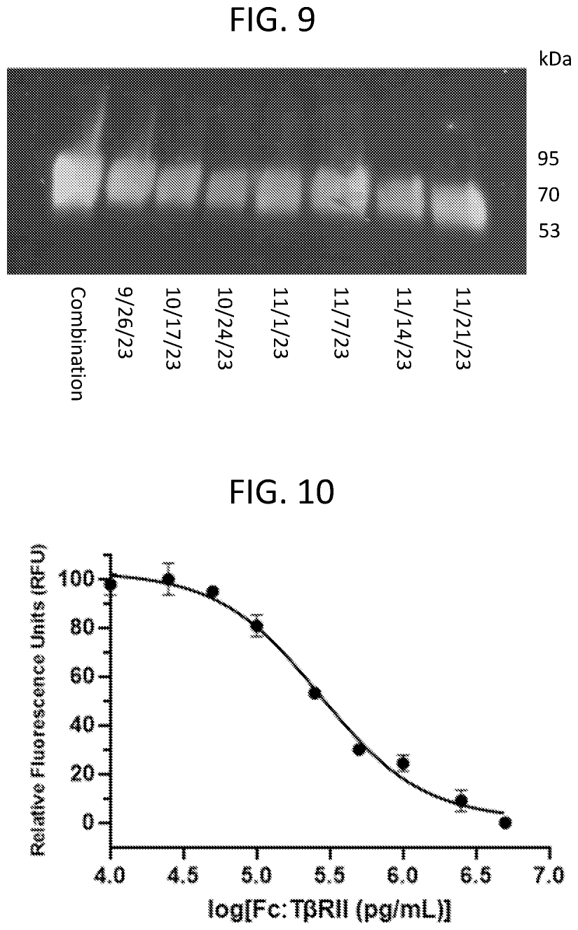

The presence of the Fc domain allows the construct to be purified from the culture supernatant using a Protein A Sepharose column. To assess purity and consistency across protein expression batches, several batches of Fc:TβRII protein expressed in HEK 293T cells were produced and purified using a protein A Sepharose column. Protein purity was found to be reliable and consistent across different batches (see, FIG. 8). The purified protein was also analyzed by Western blot, which confirmed consistent TβRII detection across the samples (see, FIG. 9).

### Example 3

### In Vitro Confirmation of TGFβ Binding

The Fc:TβRII recombinant protein (glycosylated) was tested in vitro using enzyme-linked immunosorbent assay (ELISA) to verify a selective and high binding affinity for TGFβ. A known quantity of recombinant TGFβ (500 pg/mL) was incubated with a range of concentrations of Fc:TβRII in aqueous solution at room temperature for 20 minutes. Aliquots from each sample were applied to an anti-TGFβ1 ELISA plate to measure the remaining unbound TGFβ. The ELISA results showed dose-dependent binding of the Fc:TβRII protein to TGFβ at an IC₅₀ of 0.27 μg/mL (see, FIG. 10).

### Example 4

### Conjugation of a Metal Chelator

Recombinant Fc:TβRII protein (glycosylated) was reacted with p-SCN-Bn-NOTA (which reacts with exposed lysine amines of a protein) at a molar ratio of 5:1 or 10:1 (p-SCN-Bn-NOTA: protein) for 2 hours at room temperature at pH 9. The resulting conjugated protein was purified by PD-10 size exclusion columns with PBS as the mobile phase.

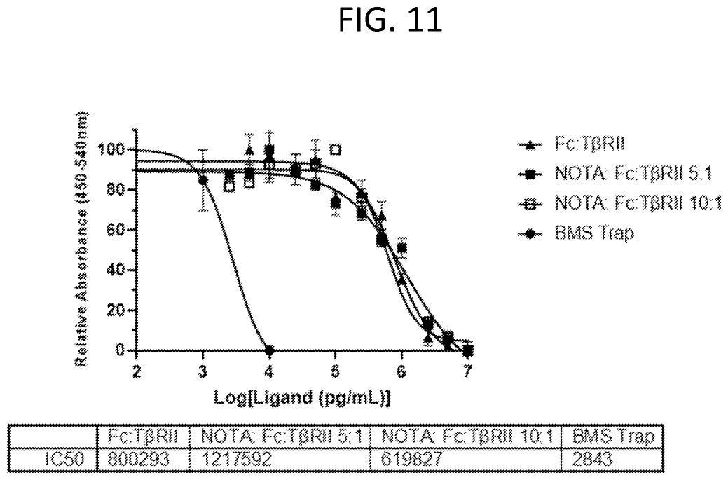

The NOTA-conjugated protein was then re-tested in vitro by ELISA to verify that conjugation of p-SCN-Bn-NOTA did not disrupt TGFβ binding. In brief, TGFβ (500 μg/mL) was incubated with a range of concentrations of unconjugated Fc:TβRII protein, conjugated Fc:TβRII protein, or control BMS trap protein in aqueous solution at room temperature for 20 minutes. Aliquots from each sample were applied to an anti-TGFβ1 ELISA plate to measure the remaining unbound TGFβ. The results show that NOTA conjugation of the Fc: TBRI protein does not disrupt TGFβ binding (see, FIG. 11).

The NOTA-conjugated proteins can be further characterized by mass spectrometry to determine the average number of NOTA moieties per protein in the product mixture.

### Example 5

### Radiolabeling of NOTA-Fc:TβRII Protein

The NOTA-Fc:TβRII conjugate is reacted with cold CuCl₂ to determine optimal radiolabeling conditions, and is radiolabeled with [⁶⁴Cu]CuCl₂ to form ⁶⁴Cu-Fc:TβRII. Radiolabeling is performed on the date of an imaging experiment.

### Example 6

### In Vivo Imaging

### Dose Titration of ⁶⁴Cu-Fc:TβRII

The optimal dose and incubation period of ⁶⁴Cu-Fc:TβRII for in vivo PET imaging is determined experimentally. Cohorts of mice are each implanted subcutaneously with tumor cells (e.g., 2×10⁵ MC38 cells in a 30 μl of PBS in the lower flank of C57BL/6 mice). Additional exemplary tumor cells and administration info is provided below in Table 1:

TABLE 1
Exemplary tumor cells and controls.

| Cancer cell lines | Route | Cell number |
|---|---|---|
| Panc02, Panc02-SIY, | s.c., orthotopic, | 1 × 10⁴-5 × 10⁶ cells |
| Panc02-NIS, | or i.v. | |
| Panc02-SIY-NIS | | |
| 3LL, 3LL-NIS | s.c. or i.v. | 1 × 10⁴-5 × 10⁶ cells |
| 4T1, 4T1-NIS | s.c., mammary | 1 × 10⁴-5 × 10⁶ cells |
| | gland or i.v. | |
| CT26, CT26-NIS | s.c. or i.v. | 1 × 10⁴-5 × 10⁶ cells |
| MC38, MC38-NIS, | s.c., orthotopic, | 1 × 10⁴-5 × 10⁶ cells |
| MC38-OVA^{APL} | or i.v. | |
| MMTV-PyMT tumor | s.c., mammary | Up to 1 × 10⁶ cells |
| preparation | r i.v | |
| MOC1 | s.c. | 1 × 10⁵-5 × 10⁶ |
| MOC1-N4-Sirius | | |
| MOC1-A2-mTagBFP2 | | |
| MOC1-Y3-EYFP | | |
| MOC1-Q4-tdTomato | | |
| MOC1-T4-Sandercyanin | | |
| MOC1-V4-mIFP | | |
| MOC1-mTagBFP2 | | |
| MOC2 | s.c. | 1 × 10⁴-5 × 10⁶ |
| MOC2-N4-Sirius | | |
| MOC2-A2-mTagBFP2 | | |
| MOC2-Y3-EYFP | | |
| MOC2-Q4-tdTomato | | |
| MOC2-T4-Sandercyanin | | |
| MOC2-V4-mIFP | | |
| MOC2-mTagBFP2 | | |
| MCA205-OVA tumor | s.c. | 1 × 10⁵-2 × 10⁶ |
| cell line | | |
| PK51975B | s.c., orthotopic | Up to 2 × 10⁶ cells/mouse |
| PK51972 | s.c., orthotopic | Up to 2 × 10⁶ cells/mouse |
| PK52003 | s.c., orthotopic | Up to 2 × 10⁶ cells/mouse |
| PK52005 | s.c., orthotopic | Up to 2 × 10⁶ cells/mouse |
| PK5L1940 | s.c., orthotopic | Up to 2 × 10⁶ cells/mouse |
| PK5L1932 | s.c., orthotopic | Up to 2 × 10⁶ cells/mouse |
| PK5L1941 | s.c., orthotopic | Up to 2 × 10⁶ cells/mouse |
| PK5L1948 | s.c., orthotopic | Up to 2 × 10⁶ cells/mouse |

After tumors reach a particular size (e.g., 25 mm²), mice are dosed intravenously (IV) by retroorbital or tail vein injection of ⁶⁴Cu-Fc:TβRII (for example, with 10 μg, 50 μg, 125 μg, or 375 μg ⁶⁴Cu-Fc:TβRII in 100 μL PBS), and imaged using small animal PET-MRI at set time intervals (e.g., at 1, 24, and 48 hr) after dosing. The images are reconstructed and standardized uptake values (SUV) for volumes of interest (VOI) in the tumor and other tissues is determined. The dosage and incubation period with optimal tumor uptake and minimal signal in other tissues is used in further experiments.

Correlation of ⁶⁴Cu-Fc:TβRII SUV with Tumor TGFβ Expression

The extent of correlation between ⁶⁴Cu-Fc:TβRII PET signal and tumor TGFβ is evaluated by imaging mice with tumors exhibiting varying degrees of TGFβ production. Exemplary tumor cell lines include oral cavity cancer MOC2, pancreatic ductal adenocarcinoma PK5L1940 (derived from spontaneous tumors generated by PDX-Cre+/− Kras(G12D)/wt p53(R172H)/wt SIYfl/− mice), and colorectal tumors MC38 and CT26. Additional examples are provided in Table 1. Cohorts of a set number of mice per tumor type (e.g., 12 per tumor type) undergo subcutaneous implantation of tumor cells from the tumor cell lines (suitable mice for CT26 is Balb/c, C57BL/6 is suitable for other listed tumor cell lines). Once tumors reach a certain size (e.g., 25 mm²), half of the animals from each cohort are dosed with the previously determined optimal dose of ⁶⁴Cu-Fc:TβRII by retroorbital injection (see, previous paragraph) and imaged using small animal PET-MRI after the optimized incubation period. The images are reconstructed and SUVs for VOIs in the tumor and normal tissues are determined.

Ex vivo analysis of tumors is performed on the imaged and non-imaged members of each cohort. Non-imaged controls are included to assess whether ⁶⁴Cu-Fc:TβRII traps TGFβ in a therapeutic capacity. TGFβ concentration in tumor lysates is determined by ELISA. TGFβ signaling is assessed by flow cytometry of Alk5, TβRII, CXCR3, CD73, CD103, and pSmad2/3. Gene expression is measured by qRT-PCR for Alk5, TβRII, PAI, IFNg, and GzmB.

### Monitoring Changes in TGFβ Following Radiation Therapy

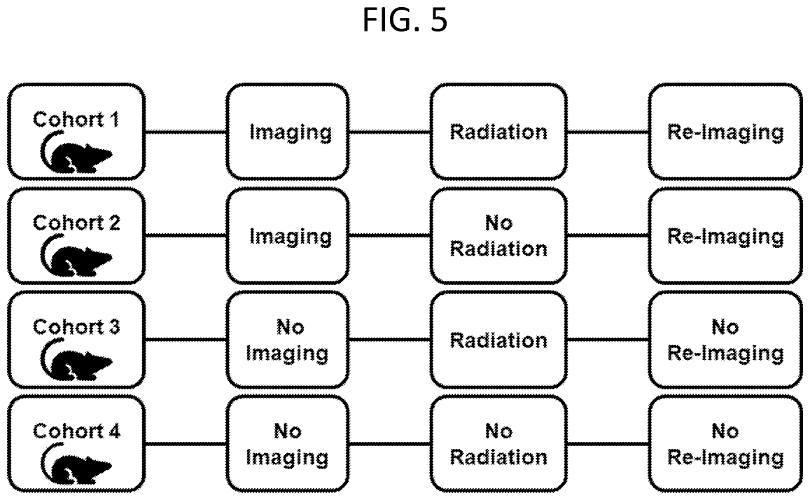

To determine whether ⁶⁴Cu-Fc:TβRII can be used to detect dynamic changes in TGFβ expression in response to radiation therapy (RT), cohorts of mice are imaged before and after RT (e.g., FIG. 5). Mice undergo tumor cell implantation (e.g., lower flank implantation of MC38 in C57BL/6 mice; see also, additional examples in Table 1). After tumors reach a certain size (e.g., 25 mm²), half of the animals are dosed with ⁶⁴Cu-Fc:TβRII by retroorbital injection and imaged using small animal PET-MRI. The images are reconstructed and SUVs for VOIs in the tumor and other tissues are determined. Subsequently, imaged and non-imaged mice undergo tumor-directed radiation using the Small Animal Radiation Research Platform (SARRP) (a device for providing tumor targeted radiation to small animals; typically, 1.8 Gy-20 Gy per fraction is used with 1-20 fractions delivered (e.g., 10 Gy×2 daily fractions)). One to fourteen days after last radiation dose (e.g., seven days after last radiation dose), “imaging” animals are re-imaged and the tumor VOIs are analyzed to evaluate changes in TGFβ expression.

Ex vivo analysis of tumors is performed on imaged and non-imaged animals. Tumor size is compared between imaged mice and non-imaged controls. TGFβ concentration in tumor lysates is determined by ELISA. TGFβ signaling is assessed by flow cytometry of Alk5, TβRII, CXCR3, CD73, CD103, and pSmad2/3. Gene expression is measured by qRT-PCR for Alk5, TβRII, PAI, IFNg, and GzmB.

It will be apparent that the precise details of the methods or compositions described herein may be varied or modified without departing from the spirit of the disclosure. We claim all such modifications and variations that fall within the scope and spirit of the claims below.

1. A recombinant molecule comprising:
(i) a recombinant protein comprising
(a) a transforming growth factor beta (TGFβ) binding domain consisting of a portion of TGFβ type II receptor (TβRII), wherein the portion specifically binds TGFβ, and
(b) an antibody Fc fragment; and
(ii) a metal ion chelator moiety covalently attached to the recombinant protein.

2. The recombinant molecule of claim 1, wherein the portion of TβRII comprises an extracellular domain of TβRII.

3. The recombinant molecule of claim 1, wherein the portion of TβRII comprises an amino acid sequence having at least 90% sequence identity to SEQ ID NO: 13, or comprises or consists of SEQ ID NO: 13.

4. (canceled)

5. The recombinant molecule of claim 1, wherein the antibody Fc fragment is an IgG2 Fc fragment; and/or
the antibody Fc fragment comprises a hinge region, CH2 domain, and/or CH3 domain.

6. (canceled)

7. The recombinant molecule of claim 1, wherein the antibody Fc fragment comprises an amino acid sequence having at least 90% sequence identity to SEQ ID NO: 5, or comprises or consists of SEQ ID NO: 5.

8. (canceled)

9. The recombinant molecule of claim 1, wherein the metal ion chelator moiety is a copper chelator.

10. The recombinant molecule of claim 1, wherein the metal ion chelator moiety is 1,4,7-triazacyclononane-1,4,7-triacetic acid (NOTA), 1,4,7,10-tetraazacyclododecane-1,4,7,10-tetraacetic acid (DOTA), or 1,4,7-triazacyclononane, l-glutaric acid-4,7-acetic acid (NODAGA).

11. The recombinant molecule of claim 1, wherein the recombinant protein:
(a) does not comprise a linker between the TGFβ binding domain and the antibody Fc domain; and/or
(b) does not comprise a fragment antigen binding (Fab) region of an antibody.

12. (canceled)

13. The recombinant molecule of claim 1, wherein the recombinant protein further comprises a spacer sequence of 8 or fewer amino acids between the portion of TβRII and the antibody Fc fragment.

14. The recombinant molecule of claim 13, wherein:
the recombinant protein consists of in N-terminus to C-terminus order, the portion of TβRII, the spacer sequence of 8 or fewer amino acids, and the IgG2 Fc domain; and
the metal ion chelator moiety is a NOTA moiety.

15. The recombinant molecule of claim 1, wherein the recombinant protein comprises an amino acid sequence having at least 90% sequence identity to SEQ ID NO: 10, or comprises or consists of SEQ ID NO: 10.

16. (canceled)

17. A composition comprising the recombinant molecule of claim 1 and a radionuclide bonded to the metal ion chelator.

18. (canceled)

19. A nucleic acid molecule encoding the recombinant protein of claim 1.

20-21. (canceled)

22. A vector comprising the nucleic acid molecule of claim 19.

23. A cell expressing the recombinant protein of claim 1.

24. A cell comprising the nucleic acid of claim 19, or a vector encoding the nucleic acid.

25-26. (canceled)

27. A kit, comprising the recombinant molecule of claim 1 or a nucleic acid encoding the recombinant molecule.

28. (canceled)

29. A method of producing the recombinant molecule of claim 1, comprising expressing the recombinant protein in a mammalian cell and subsequently attaching the covalent metal ion chelator moiety.

30-31. (canceled)

32. A method of producing the composition of claim 17, comprising incubating the recombinant molecule with the radionuclide.

33. A method of detecting transforming growth factor beta (TGFβ) in a subject, comprising:
administering the composition of claim 17 to the subject; and
detecting the composition in the subject, thereby detecting TGFβ in the subject.

34. The method of claim 33, wherein detecting comprises detecting by positron emission tomography (PET).

35-36. (canceled)
